(19) United States
(12) Patent Application Publication
(10) Pub. No.: US 2023/0181532 A1
(43) Pub. Date: Jun. 15, 2023

(54) GRANULES FOR 3D PRINTING TECHNOLOGY

(71) Applicant: BOARD OF REGENTS, THE UNIVERSITY OF TEXAS SYSTEM, Austin, TX (US)

(72) Inventors: Mohammed MANIRUZZAMAN, Austin, TX (US); Jiaxiang ZHANG, Austin, TX (US); Rishi THAKKAR, Austin, TX (US)

(73) Assignee: BOARD OF REGENTS, THE UNIVERSITY OF TEXAS SYSTEM, Austin, TX (US)

(21) Appl. No.: 17/999,299

(22) PCT Filed: May 18, 2021
(86) PCT No.: PCT/US2021/032881

§ 371 Date: Nov. 18, 2022

Related U.S. Application Data
(60) Provisional application No. 63/026,550, filed on May 18, 2020.

(51) Int. Cl. A61K 31/405; A61K 47/69; A61K 31/196; A61K 9/16; A61K 47/14

(52) U.S. Cl. CPC A61K 31/405; A61K 47/6923; A61K 31/196; A61K 9/16; A61K 47/14

(57) ABSTRACT

The present disclosure provides pharmaceutical compositions which exhibit improved flowability as measured by the angle of repose. The pharmaceutical compositions comprise an active pharmaceutical ingredient, two or more absorbents, and optionally surfactant. These pharmaceutical compositions may be used in the manufacturing of pharmaceutical dosage forms or an additive manufacturing process such as 3D selective laser sintering printing.

This application claims the benefit of priority to U.S. Provisional Application No. 63/026,550, filed on May 18, 2020, the entire content of which is hereby incorporated by reference.

### BACKGROUND

### 1. Field

The present disclosure relates generally to the field of pharmaceuticals and pharmaceutical manufacture. More particularly, it concerns compositions and methods of preparing a free-flowing pharmaceutical composition useful in an additive manufacturing application.

### 2. Description of Related Art

3D printing of personalized medication and pharmaceutical dosage form is a current and promising field of research. In the last decade, this topic of research has attracted the attention of multiple research groups and its potential has been exploited by a few pharmaceutical industries. This has led to an increase in research publications and patents in this field, 3D printing or additive manufacturing can be defined as a process of making 3-dimensional objects from a computer-aided design (CAD) model. Even though the process of 3D printing can be defined by its final product, multiple technologies work on different principles to manufacture said 3-dimensional objects. Manufacturing of 3D printed objects using a powder bed is one of these approaches. Powder bed-based 3D printing platforms have two chambers, namely the reservoir chamber and a print chamber. The reservoir chamber is filled with the powder which is used to prepare the object. This bulk of powder is transferred to the built chamber layer-by-layer where it is exposed to a stimulus in a pattern fed by the software and the digital file, forming a 3-dimensional object. Platforms operating on this mechanism include Selective laser Sintering (SLS) based 3D printing, and Binder Spraying/Jetting based 3D printing.

In order to make the abovementioned processes function smoothly and printing runs successfully, the powders should have excellent flow characteristics for both selective laser sintering and binder jetting 3D printing. In other fields of manufacturing where the powder usually comprises a single component, these processes are easy to optimize and implement. In contrast, pharmaceutical dosage forms usually contain more than one component which complicates the process for pharmaceutical 3D printing. Apart from the active pharmaceutical ingredients (API), components in a pharmaceutical dosage form include processing or performance aids. Processing aids such as binders, lubricants, glidants, fillers, and taste-masking agents, whereas the performance aids include release controlling agents, release modifying agents, and pH modifying agents. Processing a physical blend with multiple components can be challenging and can lead to several manufacturing issues such as print failure, nonuniformity of drug content, product variability (weight, % assay, dimensions), performance variability (dissolution rate, disintegration time). Therefore, there remains a need to develop and prepare pharmaceutical compositions, free-flowing pharmaceutical granules, that can be used in these promising new manufacturing techniques.

### SUMMARY

The present disclosure provides pharmaceutical compositions that may be used to prepare free-flowing pharmaceutical granules that can be used in an additive manufacturing application. In some embodiments, the disclosure provides pharmaceutical compositions comprising:

(A) an active pharmaceutical ingredient:

(B) a first absorbent:

(C) a second absorbent; and

(D) a surfactant;

wherein the pharmaceutical composition has a Carr's Index of greater than about 4 and flowability measured by the angle of repose of equal to or less than about 40.

In some embodiments, the pharmaceutical composition is present as free-flowing particles. In other embodiments, the pharmaceutical composition present as agglomerates. In some embodiments, the pharmaceutical composition comprises an amorphous active pharmaceutical ingredient. In other embodiments, the pharmaceutical composition comprises a semi-crystalline active pharmaceutical ingredient. In other embodiments, the pharmaceutical composition comprises a crystalline active pharmaceutical ingredient.

In some embodiments, the active pharmaceutical ingredient is absorbed on the first absorbent or the second absorbent. In some embodiments, the active pharmaceutical ingredient is absorbed on the first absorbent. In other embodiments, the active pharmaceutical ingredient is absorbed on the second absorbent. In some embodiments, the absorbed active pharmaceutical ingredient causes the first absorbent or the second absorbent to form an agglomeration. In some embodiments, the active pharmaceutical ingredient and the first absorbent are homogenously mixed. In other embodiments, the active pharmaceutical ingredient and the second absorbent are homogenously mixed. In some embodiments, the first absorbent and the second absorbent are homogenously mixed. In some embodiments, the active pharmaceutical ingredient, the first absorbent, and the second absorbent are homogenously mixed.

In some embodiments, the active pharmaceutical ingredient is a poorly soluble drug. In some embodiments, the active pharmaceutical ingredient is a BCS class 1 drug. In other embodiments, the active pharmaceutical ingredient is a BCS class 2 drug. In other embodiments, the active pharmaceutical ingredient is a BCS class 3 drug. In other embodiments, the active pharmaceutical ingredient is a BCS class 4 drug. In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintic, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-unnarv incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

In some embodiments, the pharmaceutical composition comprises from about 5% w/w to about 90% w/w of the active pharmaceutical ingredient. In some embodiments, the pharmaceutical composition comprises from about 10% w/w to about 80% w/w of the active pharmaceutical ingredient. In some embodiments, the pharmaceutical composition comprises from about 20% w/w to about 60% w/w of the active pharmaceutical ingredient. In some embodiments, the pharmaceutical composition comprises from about 10% w/w to about 40% w/w of the active pharmaceutical ingredient. In some embodiments, the pharmaceutical composition comprises from about 40% w/w to about 80% w/w of the active pharmaceutical ingredient.

In some embodiments, the first absorbent is a silicate. In some embodiments, the silicate is a silicate salt such as an aluminum silicate. In some embodiments, the silicate is magnesium aluminum silicate. In some embodiments, the pharmaceutical composition comprises from about 2.5% w/w to about 45% w/w of the first absorbent. In some embodiments, the pharmaceutical composition comprises from about 5% w/w to about 40% w/w of the first absorbent. In some embodiments, the pharmaceutical composition comprises from about 10% w/w to about 25% w/w of the first absorbent. In other embodiments, the pharmaceutical composition comprises from about 30% w/w to about 40% w/w of the first absorbent.

In some embodiments, the second absorbent is silica or aluminum comprising a plurality of pores. In some embodiments, the second absorbent is silica In some embodiments, In some embodiments, the second absorbent is silica comprising a plurality of pores, wherein the pores comprise a diameter between about 0.1 nm and about 50 nm. In some embodiments, the pores have a diameter between 2 nm and about 50 nm. In some embodiments, the pharmaceutical composition comprises from about 2.5% w/w to about 45% w/w of the second absorbent. In some embodiments, the pharmaceutical composition comprises from about 5% w/w to about 40% w/w of the second absorbent. In some embodiments, the pharmaceutical composition comprises from about 10% w/w to about 25% w/w of the second absorbent. In other embodiments, the pharmaceutical composition comprises from about 30% w/w to about 40/u w/w of the second absorbent. In some embodiments, the pharmaceutical composition comprises the same amount of the first absorbent and the second absorbent.

In some embodiments, the surfactant is a polysorbate derivative. In some embodiments, the surfactant is poly(ethylene glycol) derivatized polysorbate. In some embodiments, the surfactant comprises from about 10 to about 30 poly(ethylene glycol) repeating units. In some embodiments, the surfactant comprises 20 poly(ethylene glycol) repeating unit. In some embodiments, the surfactant comprises a fatty acid such as oleic acid. In some embodiments, the pharmaceutical composition comprises from about 0.5% w/w to about 20% w/w of the surfactant. In some embodiments, the pharmaceutical composition comprises from about 1% w/w to about 10% w/w of the surfactant. In some embodiments, the pharmaceutical composition comprises from about 2.5% w/w to about 7.5% w/w of the surfactant. In some embodiments, the pharmaceutical composition comprises an excipient such as a laser absorbing species. In some embodiments, the pharmaceutical composition comprises a second active pharmaceutical ingredient. In some embodiments, the pharmaceutical composition comprises a pharmaceutically acceptable polymer.

In some embodiments, the pharmaceutical composition is substantially free of any other compound. In some embodiments, the pharmaceutical composition is essentially free of any other compound. In some embodiments, the pharmaceutical composition is entirely free of any other compound. In some embodiments, the pharmaceutical composition is substantially free of any other compound other than the active pharmaceutical ingredient, the first absorbent, the second absorbent, an excipient, a second active pharmaceutical ingredient, or a pharmaceutically acceptable polymer. In some embodiments, the pharmaceutical compositions further comprise subjecting the pharmaceutical composition to milling. In some embodiments, the pharmaceutical compositions further comprise formulating the pharmaceutical composition into a unit dose. In some embodiments, the unit dose is formulated for oral delivery such as an oral delivery formulated as a tablet, capsule, or suspension.

In some embodiments, the pharmaceutical composition comprises a Carr's Index from about 5 to about 25. In some embodiments, Carr's Index is from about 5 to about 15. In some embodiments, the pharmaceutical composition comprises a surface area of greater than 100 m²/g. In some embodiments, the surface area is greater than 200 m²/g. In some embodiments, the surface area is from about 100 m²/g to about 500 m²/g. In some embodiments, the surface area is 150 m²/g to about 400 m²/g. In some embodiments, the pharmaceutical composition comprises a mean or average particle size distribution of greater than about 25 μm. In some embodiments, the mean or average particle size distribution is greater than about 50 μm. In some embodiments, the mean or average particle size distribution is from about 25 μm to about 500 μm. In some embodiments, the mean or average particle size distribution is from about 50 μm to about 250 μm. In some embodiments, the mean or average particle size distribution is from about 60 μm to about 100 μm.

In some embodiments, the pharmaceutical composition has a flowability as a function of angle of repose of less than about 35. In some embodiments, the flowability is from about 5 to about 35. In some embodiments, the flowability is from about 15 to about 30. In some embodiments, the flowability is from about 25 to about 30. In some embodiments, the pharmaceutical composition comprises a drug content uniformity of greater than about 75%. In some embodiments, the drug content uniformity is greater than 80%. In some embodiments, the drug content uniformity is from about 90% to about 110%. In some embodiments, the drug content uniformity is from about 95% to about 105%. In some embodiments, the pharmaceutical composition is formulated as granules. In some embodiments, the pharmaceutical composition comprises:
(A) about 20% w/w to about 60% w/w indomethacin;
(B) about 17.5% w/w to about 37.5% w/w magnesium aluminum silicate;
(C) about 17.5% w/w to about 37.5% w/w porous silica; and
(D) about 5% w/w of Tween® 80.

In other embodiments, the pharmaceutical composition comprises:
(A) about 20% w/w to about 80% w/w mefenamic acid;
(B) about 7.5% w/w to about 37.5% w/w magnesium aluminum silicate;
(C) about 7.5% w/w to about 37.5% w/w porous silica; and
(D) about 5% w/w of Tween® 80.

In some aspects, the present disclosure provides methods of preparing a pharmaceutical composition comprising:
(A) obtaining a mixture of an active pharmaceutical ingredient, a first absorbent, a second absorbent, and a surfactant; and
(B) subjecting the mixture to an extrusion process to obtain a pharmaceutical composition.

In some embodiments, the extrusion process is performed with a hot melt extruder. In some embodiments, the extrusion process is performed at a temperature greater than the melting point of the active pharmaceutical ingredient.

In some embodiments, the extrusion process comprises four stages. In some embodiments, the first stage comprises a first temperature from about 30° C. to about 150° C. In some embodiments, the first temperature is from about 50° C. to about 100° C. In some embodiments, the second stage comprises a second temperature from about 75° C. to about 250° C. In some embodiments, the second temperature is from about 125° C. to about 200° C. In some embodiments, the third stage comprises a third temperature from about 75° C. to about 250° C. In some embodiments, the third temperature is from about 125° C. to about 200° C. In some embodiments, the fourth stage comprises a fourth temperature from about 75° C. to about 250° C. In some embodiments, the fourth temperature is from about 125° C. to about 200° C.

In some embodiments, the extrusion process comprises a feed rate from about 1 g/min to about 25 g/min. In some embodiments, the feed rate is from about 2.5 g/min to about 10 g/min. In some embodiments, the extrusion process comprises a speed from about 10 revolutions per minute (rpm) to about 250 rpm. In some embodiments, the speed is from about 25 rpm to about 100 rpm. In some embodiments, the speed is about 50 rpm.

In some embodiments, the extrusion process has a residence time of less than 5 minutes. In some embodiments, the residence time is less than 2 minutes. In some embodiments, the residence time is less than 1 minute. In some embodiments, the extrusion process comprises an observed torque from about 20 Gm to about 200 Gm. In some embodiments, the observed torque is from about 50 Gm to about 150 Gm. In some embodiments, the observed torque is from about 60 Gm to about 100 Gm.

In some embodiments, the pharmaceutical composition is present as free-flowing particles. In other embodiments, the pharmaceutical composition present as agglomerates. In some embodiments, the pharmaceutical composition comprises an amorphous active pharmaceutical ingredient. In other embodiments, the pharmaceutical composition comprises a semi-crystalline active pharmaceutical ingredient. In other embodiments, the pharmaceutical composition comprises a crystalline active pharmaceutical ingredient.

In some embodiments, the active pharmaceutical ingredient is absorbed on the first absorbent or the second absorbent. In some embodiments, the active pharmaceutical ingredient is absorbed on the first absorbent. In other embodiments, the active pharmaceutical ingredient is absorbed on the second absorbent. In some embodiments, the absorbed active pharmaceutical ingredient causes the first absorbent or the second absorbent to form an agglomeration. In some embodiments, the active pharmaceutical ingredient and the first absorbent are homogenously mixed. In other embodiments, the active pharmaceutical ingredient and the second absorbent are homogenously mixed. In some embodiments, the first absorbent and the second absorbent are homogenously mixed. In some embodiments, the active pharmaceutical ingredient, the first absorbent, and the second absorbent are homogenously mixed.

In some embodiments, the active pharmaceutical ingredient is a poorly soluble drug. In some embodiments, the active pharmaceutical ingredient is a BCS class 1 drug. In other embodiments, the active pharmaceutical ingredient is a BCS class 2 drug. In other embodiments, the active pharmaceutical ingredient is a BCS class 3 drug. In other embodiments, the active pharmaceutical ingredient is a BCS class 4 drug. In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintic, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

In some embodiments, the pharmaceutical composition comprises from about 5% w/w to about 90% w/w of the active pharmaceutical ingredient. In some embodiments, the pharmaceutical composition comprises from about 10% w/w to about 80% w/w of the active pharmaceutical ingredient. In some embodiments, the pharmaceutical composition comprises from about 20% w/w to about 60% w/w of the active pharmaceutical ingredient. In some embodiments, the pharmaceutical composition comprises from about 10% w/w to about 40% w/w of the active pharmaceutical ingredient. In some embodiments, the pharmaceutical composition comprises from about 40% w/w to about 80% w/w of the active pharmaceutical ingredient.

In some embodiments, the first absorbent is a silicate. In some embodiments, the silicate is a silicate salt such as an aluminum silicate. In some embodiments, the silicate is magnesium aluminum silicate. In some embodiments, the pharmaceutical composition comprises from about 2.5% w/w to about 45% w/w of the first absorbent. In some embodiments, the pharmaceutical composition comprises from about 5% w/w to about 40% w/w of the first absorbent. In some embodiments, the pharmaceutical composition comprises from about 10% w/w to about 25% w/w of the first absorbent. In other embodiments, the pharmaceutical composition comprises from about 30% w/w to about 40% w/w of the first absorbent.

In some embodiments, the second absorbent is silica or aluminum comprising a plurality of pores. In some embodiments, the second absorbent is silica. In some embodiments, In some embodiments, the second absorbent is silica comprising a plurality of pores, wherein the pores comprise a diameter between about 0.1 nm and about 50 nm. In some embodiments, the pores have a diameter between 2 nm and about 50 nm. In some embodiments, the pharmaceutical composition comprises from about 2.5% w/w to about 45% w/w of the second absorbent. In some embodiments, the pharmaceutical composition comprises from about 5% w/w to about 40% w/w of the second absorbent. In some embodiments, the pharmaceutical composition comprises from about 10% w/w to about 25% w/w of the second absorbent. In other embodiments, the pharmaceutical composition comprises from about 30% w/w to about 40% w/w of the second absorbent. In some embodiments, the pharmaceutical composition comprises the same amount of the first absorbent and the second absorbent.

In some embodiments, the surfactant is a polysorbate derivative. In some embodiments, the surfactant is poly(ethylene glycol) derivatized polysorbate. In some embodiments, the surfactant comprises from about 10 to about 30 poly(ethylene glycol) repeating units. In some embodiments, the surfactant comprises 20 poly(ethylene glycol) repeating unit. In some embodiments, the surfactant comprises a fatty acid such as oleic acid. In some embodiments, the pharmaceutical composition comprises from about 0.5% w/w to about 20% w/w of the surfactant. In some embodiments, the pharmaceutical composition comprises from about 1% w/w to about 10% w/w of the surfactant. In some embodiments, the pharmaceutical composition comprises from about 2.5% w/w to about 7.5% w/w of the surfactant. In some embodiments, the pharmaceutical composition comprises an excipient such as a laser absorbing species. In some embodiments, the pharmaceutical composition comprises a second active pharmaceutical ingredient. In some embodiments, the pharmaceutical composition comprises a pharmaceutically acceptable polymer.

In some embodiments, the pharmaceutical composition is substantially free of any other compound. In some embodiments, the pharmaceutical composition is essentially free of any other compound. In some embodiments, the pharmaceutical composition is entirely free of any other compound. In some embodiments, the pharmaceutical composition is substantially free of any other compound other than the active pharmaceutical ingredient, the first absorbent, the second absorbent, an excipient, a second active pharmaceutical ingredient, or a pharmaceutically acceptable polymer. In some embodiments, the pharmaceutical compositions further comprise subjecting the pharmaceutical composition to milling. In some embodiments, the pharmaceutical compositions further comprise formulating the pharmaceutical composition into a unit dose. In some embodiments, the unit dose is formulated for oral delivery such as an oral delivery formulated as a tablet, capsule, or suspension.

In some embodiments, the pharmaceutical composition comprises a Carr's Index from about 5 to about 25. In some embodiments, Carr's Index is from about 5 to about 15. In some embodiments, the pharmaceutical composition comprises a surface area of greater than 100 m²/g. In some embodiments, the surface area is greater than 200 m²/g. In some embodiments, the surface area is from about 100 m²/g to about 500 m²/g. In some embodiments, the surface area is 150 m²/g to about 400 m²/g. In some embodiments, the pharmaceutical composition comprises a mean or average particle size distribution of greater than about 25 μm. In some embodiments, the mean or average particle size distribution is greater than about 50 μm. In some embodiments, the mean or average particle size distribution is from about 25 μm to about 500 μm. In some embodiments, the mean or average particle size distribution is from about 50 μm to about 250 μm. In some embodiments, the mean or average particle size distribution is from about 60 μm to about 100 μm.

In some embodiments, the pharmaceutical composition has a flowability as a function of angle of repose of less than about 40. In some embodiments, the flowability is from about 5 to about 40. In some embodiments, the flowability is from about 15 to about 35. In some embodiments, the flowability is from about 20 to about 30. In some embodiments, the pharmaceutical composition comprises a drug content uniformity of greater than about 75%. In some embodiments, the drug content uniformity is greater than 80%. In some embodiments, the drug content uniformity is from about 90% to about 110%. In some embodiments, the drug content uniformity is from about 95% to about 105%. In some embodiments, the pharmaceutical composition is formulated as granules. In some embodiments, the pharmaceutical composition comprises:
(A) about 20% w/w to about 60% w/w indomethacin;
(B) about 17.5% w/w to about 37.5% w/w magnesium aluminum silicate;
(C) about 17.5% w/w to about 37.5% w/w porous silica; and
(D) about 5% w/w of Tween® 80.

In other embodiments, the pharmaceutical composition comprises:
(A) about 20% w/w to about 80% w/w mefenamic acid;
(B) about 7.5% w/w to about 37.5% w/w magnesium aluminum silicate;
(C) about 7.5% w/w to about 37.5% w/w porous silica; and
(D) about 5% w/w of Tween® 80.

In still yet another aspect, the present disclosure provides methods of preparing a unit dose comprising:
(A) obtaining a pharmaceutical composition described herein; and
(B) subjecting the pharmaceutical composition to an additive manufacturing process to obtain a unit dose.

In some embodiments, the additive manufacturing process is a 3D printing process. In some embodiments, the additive manufacturing process is an additive manufacturing layer process. In some embodiments, the additive manufacturing process is selective layer sintering. In some embodiments, the unit dose is formulated in a manner to be directly administered to a patient without further processing.

In still yet another aspect, the present disclosure provides pharmaceutical compositions prepared for the methods described herein.

In another aspect, the present disclosure provides methods of treating a disease or disorder in a patient in need thereof comprising administering to the patient a therapeutically effective amount of a pharmaceutical composition described herein, wherein the active pharmaceutical ingredient is effective to treat the disease or disorder.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A & 1B show the process schematic for the manufacturing of the granules for Examples A, B, and C.

FIG. 2 shows the thermal characterization (differential scanning calorimetry) of the manufactured granules where the processing conditions were maintained such that the drug was completely rendered amorphous after the process. (Examples A, B, and C).

FIG. 3 shows the solid-state characterization (powder X-ray diffraction) of the manufactured granules where the processing conditions were maintained such that the drug was completely rendered amorphous after the process. (Examples A, B, and C)

FIG. 4 shows the thermal characterization (differential scanning calorimetry) of the manufactured 3D printed tablets where the processing conditions were maintained such that the drug remained completely amorphous after the process. (Example D)

FIG. 5 shows the solid-state characterization (powder X-ray diffraction) of the manufactured 3D printed tablet where the processing conditions were maintained such that the drug remained amorphous after the process. (Example D)

FIG. 6 shows the solid-state characterization (polarized light microscopy) of the manufactured granules printed tablet where the processing conditions were maintained such that the drug remained amorphous after the process. (Example D)

FIG. 7 shows the solid-state characterization (polarized light microscopy) of the manufactured 3D printed tablet where the processing conditions were maintained such that the drug remained amorphous after the process. (Example D)

FIG. 8 Shows the photographs of the manufactured 3D printed tablet with porous morphology where the processing conditions were maintained such that the drug remained amorphous after the process. (Example D)

FIGS. 9A-9D show (FIG. 9A) SEM of unprocessed composition (left) and processed (right) Example D depicting the absorption of the drug on the carrier/absorbent. (FIG. 9B) SEM of unprocessed composition (left) and processed (right) Example E depicting the absorption of the drug on the carrier/absorbent. (FIG. 9C) SEM of unprocessed composition (left) and processed (right) Example F depicting the absorption of the drug on the carrier/absorbent. (FIG. 9D) SEM of unprocessed composition (left) and processed (right) Example G depicting the absorption of the drug on the carrier/absorbent.

FIGS. 10A-10D show the process monitoring of CIELAB yellow-blue color space coordinate (b*), custom selected wavelength (600-700 nm), yellowness index (‘E313-00 YI’ which is supposed to trend with b*), the wavelength of maximum reflectance over the measured region (PWL), and reflectance value at the PWL (Peak) with UV-Visible reflectance probe at different extrusion temperatures at A) at 140° C. B) at 145° C. C) at 150° C. D) at 155° C.

FIGS. 11A1-3-11D1-3 show A) Digital microscopy images, A-1) Indomethacin crystals, A-2) Physical Mixture-I, A-3) Processed granules; B) Polarized light microscopy (530 nm compensator) images, B-1) Indomethacin crystals, B-2) Physical Mixture-I, B-3) Processed granules; C) Polarized Light Microscopy (dark mode), C-1) Indomethacin crystals, C-2) Physical Mixture-I, C-3) Processed granules; D) Scanning Electron Microscopy, D-1) highlighted Indomethacin crystal, D-2) Physical Mixture-I, D-3) Processed granules.

FIGS. 12A & 12B show flow-through orifice ‘weight versus time’ plots for three different orifice diameters (10, 15, 25 mm) (FIG. 12A) Extruded granules (FIG. 12B) PA-12 (LS reference material).

FIG. 13 shows the morphology of LS 3D printed indomethacin tablets using HME based granulation technique.

FIG. 14 shows the powder X-ray diffraction analysis of indomethacin, excipients, physical mixtures, extruded granules, and 3D printed tablets.

FIG. 15 shows the modulated differential scanning calorimetry of IND, excipients, physical mixtures, extruded granules, and 3D printed tablets.

FIGS. 16A & 16B shows the Fourier transform infrared spectroscopy of IND, excipients, physical mixtures, extruded granules, and 3D printed tablets.

FIGS. 17A-17D show the FT-Raman spectra of A) extruded granules B) Pre-extrusion physical mixture (PM-I) C) Shift in ‘carbonyl stretching’ region because of amorphous conversion post extrusion D) Carbonyl stretching region of crystalline IND in PM-I.

FIGS. 18A & 18B show (FIG. 18A) pH shift dissolution study for pure crystalline IND, PM-I, PM-II, Granules, LS 3D printed tablets, hot melt-extruded reference amorphous solid dispersion. (FIG. 18B) Dissolution study for pure crystalline IND, PM-I, PM-II, LS 3D printed tablets, hot melt-extruded reference amorphous solid dispersion at pH 2.

FIG. 19 shows powder X-ray diffraction analysis of indomethacin, excipients, physical mixtures, extruded granules, and 3D printed tablets.

FIG. 20 shows the positions of the parts (referred to as printlet or tablet in this manuscript) in the build platform with a maximum build volume 110×110×110 and a recommended build volume of 90×90×90 (units are in mm).

FIG. 21 shows the schematic of granule manufacturing and 3D printing process

### DESCRIPTION OF ILLUSTRATIVE EMBODIMENTS

In some aspects of the present disclosure, the pharmaceutical compositions provided herein may exhibit flowability properties that allow them to be used in additive manufacturing methods. The active pharmaceutical ingredient may be used to act as a binder between the absorbent. The process of manufacturing the pharmaceutical formulation may comprise of melting and absorption before cooling and recrystallization of the active pharmaceutical ingredient leading to an agglomeration of the particles. The agglomeration of the particles often results from intermolecular forces holding the mixture together to produce free-flowing granules. These methods may be used with a wide array of different additive manufacturing platforms. The resulting pharmaceutical composition from the process may exhibit a high surface area, an average particle size distribution of greater than 60 μm, a flowability (angle of repose) that is greater than 25, a Carr's index within 5-15, drug content uniformity that is greater than 80% while maintaining a drug loading of greater than 10%. These compositions may be used in additive manufacturing methods such as selective laser sintering to obtain a 3D printed pharmaceutical composition. Methods of preparing these compositions are described in more detail therein.

### I. PHARMACEUTICAL COMPOSITIONS

In some aspects, the present disclosure provides pharmaceutical compositions containing an active pharmaceutical ingredient or a pharmaceutically acceptable salt, ester, derivative, analog, pro-drug, or solvates thereof, two or more absorbents, and optionally one or more surfactants. These compositions may exhibit one or more free-flowing properties such as having a flowability as measured by the angle of repose of less than 25. These compositions may exhibit a flowability as measured by the angle of repose of less than about 25, less than about 27.5, less than about 30, less than about 32.5, less than about 35, less than about 37.5, or less than about 40. The flowability may be from about 25 to about 40, or from about 25 to about 30. The flowability may be from about 2, 4, 5, 6, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein. The flowability of the pharmaceutical composition is measured by, The simplest method for the determination of the angle of repose is the “poured” angle. A funnel with a wide outlet is affixed at a distance of 10 cm above the bench, where a piece of paper is placed directly beneath the funnel. The granules are added while the funnel is closed. The contents flow through and collect on the paper. The diameter of the cone (D) and two opposite sides (l₁+l₂) are measured with rulers. The angle of repose (θ) is calculated from the equation arc cos[D/(l₁+l₂)]. The relationship between flow properties and angle of repose has been established. When the angle of repose is less than 25 degrees, the flow is said to be excellent; on the other hand, if the angle of repose is more than 40 degrees, the flow is considered to be poor. These pharmaceutical compositions may be present as agglomerations and used in either a batch, semi-continuous, continuous manufacturing process. The active pharmaceutical ingredient may act as a binder between the absorbent particles within the pharmaceutical composition.

In other aspects, the present pharmaceutical compositions may exhibit a mean or average particle size distribution greater than 25 μm, greater than 50 μm, or greater than 60 μm. In some embodiments, the pharmaceutical compositions exhibit a mean or average particle size from about 25 μm to about 500 μm, 30 μm to about 400 μm, 35 μm to about 350 μm, 40 μm to about 300 μm, 50 μm to about 250 μm, 50 μm to about 200 μm, 50 μm to about 150 μm, 55 μm to about 125 μm, or from about 60 μm to about 100 μm. The mean or average particle size of the pharmaceutical composition comprises from about 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 150 μm, 175 μm, 200 μm, 250 μm, 300 μm, 350 μm, 400 μm, 450 μm, 500 μm, 600 μm, 700 μm, 800 μm, 900 μm, to about 1000 μm, or any range derivable therein. The mean or average particle size of the pharmaceutical composition may be determined by mesh analysis using a sonic sifter. The particle size distribution of the dried granules can also be determined by a dry laser diffraction technique or scanning electron microscopy. Furthermore, the pharmaceutical composition may have a specific surface area that is greater than 50 m²/g, greater than 100 m²/g, greater than 150 m²/g, greater than 200 m²/g, or greater than 250 m²/g, or greater than 300 m²/g. The pharmaceutical composition may have a specific surface area from about 50 m²/g to about 5,000 m²/g, from about 100 m²/g to about 2,000 m²/g, or from about 200 m²/g to about 500 m²/g. The pharmaceutical composition may comprise a specific surface area from about 50 m²/g, 75 m²/g, 100 m²/g, 150 m²/g, 175 m²/g, 200 m²/g, 225 m²/g, 250 m²/g, 275 m²/g, 300 m²/g, 400 m²/g, 500 m²/g, 600 m²/g, 700 m²/g, 750 m²/g, 800 m²/g, 900 m²/g, 1,000 m²/g, 2,000 m²/g, 5,000 m²/g, to about 10,000 m²/g, or any range derivable therein. The specific surface area may be measured using the Brunauer, Emmett, and Teller (BET) method.

In some aspects, the pharmaceutical composition may exhibit a drug content uniformity greater than 75%, greater than 80%, greater than 85%, greater than 90%, greater than 95%, or greater than 99%. In some embodiments, the drug content uniformity is from about 80% to about 120%, from about 85% to about 115%, from about 90% to about 110%, or from about 95% to about 105%. The drug content uniformity may be from about 75%, 80%, 85%, 90%, 95%, 100%, 105%, 110%, 115%, 120%, to about 125%, or any range derivable therein. The drug content uniformity of the pharmaceutical composition may be determined by taking samples from three regions of the bulk mass of the manufactured granules. The drug may be extracted using appropriate solvents and analyzed using spectrophotometric tools such as UV-Vis spectrophotometer, or high-performance liquid chromatography (HPLC). The uniformity will be reported as the mean percent of the expected content±standard deviation.

In some aspects, the pharmaceutical composition may exhibit a Carr's Index is from about 5 to about 28, from about 5 to about 25, from about 5 to about 21, from about 5 to about 15, or from about 5 to about 10. The Carr's Index may be from about 2, 4, 5, 6, 7, 8, 9, 10, 11, 12, 13, 14, 15, 16, 17, 18, 19, 20, 21, 22, 23, 24, 25, 28, 30, 32, 35, 38, to about 40, or any range derivable therein. Carr's Index of the pharmaceutical composition may be determined by tapped density which is measured after a powder sample is subjected to mechanically tapping. The measurement procedure for bulk density and tapped density can be found in the US Pharmacopeia. Bulk density and tapped density can be used to calculate the carr's compressibility index and Hausner ratio, which are measures of the propensity of a powder to flow and be compressed:

$$Compressibility index ( \% ) = \frac{tapped density - bulk density}{tapped density} \times 100$$

$$Hausner ' s ratio = \frac{Tapped density}{bulk density}$$

In some aspects, the present pharmaceutical composition may be exhibit compressibility that makes the composition useful for the production of pharmaceutical dosage forms such as oral forms like capsules or tablets. The pharmaceutical composition may also be used in a powder-based additive manufacturing application such as selective laser sintering based 3D printing. These 3D printing platforms may be used in pharmaceutical manufacturing and patient-specific personalized therapy to produce on-demand pharmaceutical compositions.

A. Active Pharmaceutical Ingredient

The pharmaceutical compositions described herein comprise an active pharmaceutical ingredient. The pharmaceutical compositions described herein contain an active pharmaceutical ingredient in an amount between about 5% to about 95% w/w, between about 10% to about 90% w/w, between about 20% to about 80% w/w, or between about 25% to about 50% w/w of the total composition. In some embodiments, the amount of the active pharmaceutical ingredient is from about 5%, 10%, 20%, 22%, 24%, 25%, 26%, 28%, 30%, 32%, 34%, 35%, 36%, 38%, 40%, 42%, 44%, 45%, 50%, 55%, 60%, 65%, 70%, 75%, 80%, 90%, to about 95% w/w or any range derivable therein. In some embodiments, the pharmaceutical composition is substantially, essentially, or entirely free of any other active pharmaceutical ingredient.

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the intestinal cell layer. According to the BCS, drug substances are classified as follows: Class I—High Permeability, High Solubility; Class II—High Permeability, Low Solubility; Class III—Low Permeability, High Solubility; and Class IV—Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconazole, Lopinavir, Mebendazole, Nelfinavir, Nevirapine. Niclosamide, Praziquantel, Pyrantel, Pyrimethamine, Quinine, and Ritonavir. Antineoplastic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone. Telmisartan, Ticlopidine, Valsartan, Verapamil, Warfarin. Central Nervous System Agents such as Acetaminophen. Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam. Meloxicam, Metaxalone, Methylphenidate, Metoclopramide, Nicergoline, Naproxen, Olanzapine, Oxcarbazepine. Phenytoin, Quetiapine Risperidone, Rofecoxib, and Valproic acid. Dermatological Agents such as Isotretinoin—Endocrine and Metabolic Agents such as Dexamethasone, Danazol. Epalrestat, Gliclazide, Glimepiride, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, i pyridostigmine, sodium cloxacillin, thiamine, benzimidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone. BI-639667, rifabutin, paclitaxel, curcumin, etoposide, neomycin, methotrexate, atazanavir sulfate, Aprepitant, amphotericin B, amiodarone hydrochloride, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other API that are of specific consideration are those that are high melting point drugs such as a drug that has a melting point of greater than 200° C. Alternatively, the API used herein may have a melting point from about 25° C. to about 1,000° C., from about 100° C. to about 750° C., or from about 200° C. to about 500° C. In particular, the melting point may be greater than 200° C., 250° C., 300° C., 400° C., 500° C., 300° C., 700° C. 750° C., 800° C. 900° C., or 1,000° C.

In some aspects, the present methods may be used to formulate one or more poorly soluble API such as deferasirox, etravirine, indomethacin, posaconazole, and ritonavir. Etravirine is a neutral active pharmaceutical ingredient and may be used as a model for other neutral API. Deferasirox and indomethacin is a weak acid API and may be used as a model for other weak acid APIs. Posaconazole, itraconazole, and ritonavir are weak base APIs and may be used as models for other weak base APIs.

Suitable API may be any poorly water-soluble, biologically API or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the API may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glybunde, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hvoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxiflIline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the API may be busulfan, taxane, or other anticancer agents; alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole, and c) thiazoles such as abafungin. Other API that may be used with this approach include, but are not limited to, hyperthyroid drugs such as carbimazole, anticancer agents like cytotoxic agents such as epipodophyllotoxin derivatives, taxanes, bleomycin, anthracyclines, as well as platinum compounds and camptothecin analogs. The following API may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The API may also include a psychiatric agent such as an antipsychotic, anti-depressive agent, or analgesic and/or tranquilizing agents such as benzodiazepines. The API may also include a consciousness level-altering agent or an anesthetic agent, such as propofol. The present compositions and the methods of making them may be used to prepare a pharmaceutical composition with the appropriate pharmacokinetic properties for use as therapeutics.

B. Excipients

In some aspects, the present disclosure comprises one or more excipients formulated into pharmaceutical compositions including a first and second absorbent. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

1. Absorbents

In some aspects, the pharmaceutical composition may further comprise one or more inorganic or organic material that have a high surface area where the active pharmaceutical ingredient may be absorbed onto the material. These components of the pharmaceutical compositions may be referred to as an absorbent. Without wishing to be bound by any theory, it is believed that the active pharmaceutical ingredient is retained on the surface of the absorbent. Then once absorbed onto the absorbent the active pharmaceutical ingredient may then cool or recrystallize to form an agglomerate with the surrounding particles to form a granule. The absorbent may be either an inorganic or an organic compound. In some embodiments, the organic absorbent is an organic polymer such as cellulose or another pharmaceutically acceptable polymer. In other embodiments, the organic absorbent is a lipid. In other aspects, the absorbent may be an inorganic absorbent such as silica or silicate composition. The absorbent may comprise either a high porosity and a high surface area. The porosity of the absorbent may be from about 1% to about 80%, from about 2% to about 60%, from about 5% to about 50%, or from about 10% to about 45%. The porosity of the absorbent may be from about 1%, 2%, 3%, 4%, 5%, 10%, 15%, 20%, 25%, 30%, 35%, 40%, 45%, 50%, 55%, 60%, 65%, 70%, 75%, to about 80%, or any range derivable therein. The absorbent may further comprise a high specific surface area as measured by the Brunauer, Emmett, and Teller (BET) specific surface area. The specific surface area of the absorbent may be greater than 50 m²/g, greater than 100 m²/g, greater than 150 m²/g, greater than 200 m²/g, or greater than 250 m²/g, or greater than 300 m²/g. The absorbent may have a specific surface area from about 50 m²/g to about 5,000 m²/g, from about 100 m²/g to about 2,000 m²/g, or from about 200 m²/g to about 500 m²/g. The absorbent may comprise a specific surface area from about 50 m²/g, 75 m²/g, 100 m²/g, 150 m²/g, 175 m²/g, 200 m²/g, 225 m²/g, 250 m²/g, 275 m²/g, 300 m²/g, 400 m²/g, 500 m²/g, 600 m²/g, 700 m²/g, 750 m²/g, 800 m²/g, 900 m²/g, 1,000 m²/g, 2,000 m²/g, 5,000 m²/g, to about 10,000 m²/g, or any range derivable therein.

In some embodiments, either the first absorbent or the second absorbent is silica. Silica has a chemical formula of SiO₂ and may show multiple different polymorphic forms. These polymorphic forms include α-quartz, β-quartz, α-tridymite, β-tridymite, α-cristobalite, β-cristobalite, keatite, moganite, coesite, stishovite, seifertite, melanophlogite, fibrous W-silica, or 2D silica. In some embodiments, the silica comprises one or more pores that pass through the silica. The pores within the silica may have a diameter of less than 100 nm. In some embodiments, the diameter of the pores in the silica may be less than 50 nm. The diameter of the pores may be mesoporous such that the silica is mesoporous silica with diameters from 2 nm to about 50 nm. In other embodiments, the silica may be microporous silica with a diameter of less than 2 nm. In some embodiments, the diameter of the pores in the silica may be from about 0.1 nm, 0.5 nm, 1 nm, 2 nm, 3 nm, 4 nm, 5 nm, 10 nm, 15 nm, 20 nm, 25 nm, 30 nm, 35 nm, 40 nm, 45 nm, 50 nm, 60 nm, 70 nm, 80 nm, 90 nm, to about 100 nm, or any range derivable therein.

In another embodiment, the pharmaceutical composition may comprise a first absorbent or a second absorbent, wherein the absorbent is a silicate. The silicate may comprise a formula of silicon and oxygen comprising a general formula of [SiO₄₋ₓ^{4-2x-}]ₙ, wherein x is greater than or equal to 0 but less than 2. These silicates may be either a salt or an ester of an alkyl group. The salt may comprise a counterion of either a transition metal, a metalloid, an alkali earth metal, or an alkali metal. The counterion may be either sodium, potassium, magnesium, calcium, and aluminum. The silicate may further comprise one or more or more aluminum ions wherein the aluminum is a tetravalent ion that replaces one or more of the silicon. These materials are also known as aluminosilicate. Silicates may be either orthosilicate, metasilicate, pyrosilicate, or a polymeric silicate such as chains, rings, double chains, or sheets. The silicate may be formulated in manners that comprise one or more pores. The pores within the silicate may have a diameter of less than 100 nm. In some embodiments, the diameter of the pores in the silicate may be less than 50 nm. The diameter of the pores may be mesoporous such that the silicate is a mesoporous silicate with diameters from 2 nm to about 50 nm. In other embodiments, the silicate may be microporous silicate with a diameter of less than 2 nm. In some embodiments, the diameter of the pores in the silicate may be from about 0.1 nm, 0.5 nm, 1 nm, 2 nm, 3 nm, 4 nm, 5 nm, 10 nm, 15 nm, 20 nm, 25 nm, 30 nm, 35 nm, 40 nm, 45 nm, 50 nm, 60 nm, 70 nm, 80 nm, 90 nm, to about 100 nm, or any range derivable therein.

Furthermore, the pharmaceutical composition described herein have a concentration of each of the absorbents ranging from about 1% to about 49% w/w. In some embodiments, the amount of each absorbent is from about 1% to about 49% w/w, from about 2% to about 47.5% w/w, 2.5% to about 45% w/w, or 10% to about 40% w/w. The amount of each absorbent may be from about 1%, 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, to about 49%, or any range derivable therein. Each of the absorbents may be present in the same amounts. Alternatively, the amount of each absorbent is present in a different amount. In some embodiments, the pharmaceutical composition is substantially, essentially, or entirely free of any other absorbents.

2. Surfactant

In some aspects, the present disclosure provides pharmaceutical compositions comprising one or more surfactants. As used herein, the term “surfactant” refers to a compound that exhibits amphiphilic character and reduces the surface tension of a solvent, particularly water. Surfactants can generally be classified into four categories: cationic, anionic, zwitterionic, or non-ionic. While it is contemplated that any of these surfactants may be used in the present compositions, non-ionic surfactants show particular promise. Cationic surfactants include, but are not limited to, amines with long alkyl chains and are protonated at a physiologically relevant pH or permanently charged quaternary ammonium salts such as cetrimonium bromide, cetylpyridinium chloride, benzalkonium chloride, benzethonium chloride, dimethyldioctadecylammonium chloride, or dioctadecyldimethylammonium bromide. Some non-limiting examples of anionic surfactants include sulfate, sulfonate, or phosphate esters such as docusate, perfluorooctanesulfonate, perfluorobutanesulfonate, alkyl-aryl ether phosphates, or alkyl ether phosphate or carboxylate esters including aliphatic carboxylates such as fatty acids and derivatives thereof. Other examples of zwitterionic surfactants including phospholipids such as phosphatidylserine, phosphatidylcholine, phosphatidylethanolamine, or sphingomyelins, sultaines such as CHAPS and cocamidopropyl hydroxysultaine, or betaine such as cocamidopropyl betaine. Finally, some non-limiting examples of nonionic surfactants include PEG alkyl ethers, polypropylene glycol ethers, glucoside alkyl ethers, PEG alkylaryl ethers such as Triton® and nonoxynol, simple alkyl esters of glycerol such as glycerol laurate, polysorbates such as Tween®, Sorbitan alkyl esters such as Span, or poloxamer and other block copolymers of polyethylene glycol and polypropylene glycol. In some embodiments, the surfactants used in the present pharmaceutical compositions contain one or more polyethylene glycol or polypropylene glycol polymers such as Tween, Capryol, Labrafil, or Labrasol.

In some embodiments, the surfactant is a compound with a PEG polymer with a molecular weight from about 100 to about 4000 daltons, from about 100 to about 1000 daltons, from about 100 to about 500 daltons, or from about 100, 200, 300, 400, 500, 600, 700, 800, 900, 100, 1250, 1500, 1750, 2000, 2500, 3000, 3500, or about 4000 daltons. In other embodiments, the surfactant comprises one or more polyethylene glycol polymers with the polyethylene glycol repeating units comprises at the total number from 5 to 50 repeating units. The number of repeating units in the polyethylene glycol components comprises from 5, 6, 8, 10, 12, 14, 15, 16, 18, 20, 22, 24, 25, 26, 28, 30, 32, 34, 35, 36, 38, to 40 repeating units. The surfactant may further comprise one or more lipid or oil elements. The lipid or oil element may be a fatty acid, a triglyceride, an ester of fatty acid, or mixtures thereof. The term lipid includes fatty acids which are a group of aliphatic saturated or unsaturated carboxylic acids. The chains are usually, unbranched and have 6 to 30, preferably 8 to 22, and in particular 8 to 18, carbon atoms. Some non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotic acid, and melissic acid Additionally, the term includes unsaturated fatty acids may be unsaturated one or more times, in particular, unsaturated once, twice, three times, four times, five times or six times. Some non-limiting examples of singly unsaturated fatty acids include palmitoleic acid, oleic acid, and erucic acid, of doubly unsaturated fatty acids, include sorbic acid and linoleic acid, of triply unsaturated fatty acids, including linolenic acid and eleostearic acid, of quadruply unsaturated fatty % acids including arachidonic acid, of quintuply % unsaturated fatty acids include clupanodonic acid, and of sextuply unsaturated fatty acids include docosahexaenoic acid The surfactant may also further comprise a sugar unit. The sugar unit may be ribose or sorbitan. In some embodiments, the surfactant may be a polysorbate such as a polysorbate 20, polysorbate 40, polysorbate 60, or polysorbate 80.

In some aspects, the amount of the surfactant is from about 1% to about 20% w/w, from about 2% to about 10% w/w, from about 2% to about 8% w/w, or from about 2% to about 4% w/w. The amount of the surfactant comprises from about 1%, 1.25%, 1.5%, 1.75%, 2%, 3%, 4%, 5%, 6%, 7%, 8%, 9%, 10%, 12%, 14%, 15%, 16%, 18%, to about 20% w/w, or any range derivable therein, of the total pharmaceutical composition. In one embodiment, the amount of the surfactant is at 2% to 5% w/w of the total weight of the pharmaceutical composition. In some embodiments, the pharmaceutical composition is substantially, essentially, or entirely free of any other surfactants.

3. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active pharmaceutical ingredient in a given solution so that the particles formed contain more than one active pharmaceutical ingredient.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polvoxyethvlated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glvcolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active pharmaceutical ingredient in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethyleneglycols. Labrasol®, pol vinyl alcohols, polyvinyl pyrrolidones, and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing agent, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In some aspects, the amount of the excipient in the pharmaceutical composition is from about 0.5% to about 20% w/w, from about 1% to about 10% w/w, from about 2% to about 8% w/w, or from about 3% to about 7% w/w. The amount of the excipient in the pharmaceutical composition comprises from about 0.5%, 1%, 1.5%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 4% to 6% w/w of the total weight of the pharmaceutical composition.

### II. ADDITIVE MANUFACTURING PLATFORMS

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

In other aspects, the pharmaceutical compositions described herein may also be used in an additive manufacturing platform. Some of the additive manufacturing platforms that may be used herein include 3D printing such as selective laser sintering or selective laser melting. Alternatively, a method such as stereolithography or fused deposition modeling may be used to obtain the final pharmaceutical composition. The pharmaceutical compositions described herein may be used these processes and exhibit a flowability as measured by the angle of repose of less than 25. The pharmaceutical composition may have a flowability of less than 25, less than 26, less than 27, less than 28, less than 29, less than 30, less than 32.5, less than 35, or less than 40.

These pharmaceutical compositions may be processed through laser sintering wherein a laser is aimed at a specific point on the pharmaceutical composition such that material is bound together to create a solid form. The laser is passed over the surface in a sufficient amount of time and sufficient location to produce the desired dosage form. The method relates to the use of the laser-based upon the power of the laser such as the peak laser power rather than the laser duration. The method often will make use of a pulsed laser. The laser used in these methods often is a high power laser such as a carbon dioxide laser. The process builds up the dosage form using cross-sections of the material through multiple scanning passes over the material. Additionally, the chamber of the 3D printer device may also be preheated to a temperature just below the melting point of the pharmaceutical composition such as the melting point of the composition as a whole or the active agent, the absorbent, or the surfactant. Furthermore, the method may be used without the need for a secondary feeder of material into the chamber of the device.

### III. DEFINITIONS

The use of the word “a” or “an”, when used in conjunction with the term “comprising” in the claims and/or the specification, may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”. “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions.” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, to alleviate signs or symptoms of the disease. Desirable effects of treatment include decreasing the rate of disease progression, ameliorating or palliating the disease state, and remission or improved prognosis. Alleviation can occur before signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of the cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active pharmaceutical ingredient in the composition is measured by powder x-ray diffraction.

A “poorly soluble drug” refers to a drug that meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10.000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature that is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active pharmaceutical ingredient to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Formulations

### Example A

TABLE 1
Formulation composition
Formulation 1

| Ingredient | Purpose | % weight |
|---|---|---|
| Indomethacin | API | 10 |
| Magnesium aluminum silicate | Absorbent | 42.5 |
| Porous silica | Absorbent | 42.5 |
| Tween 80 ® | Surfactant | 5 |

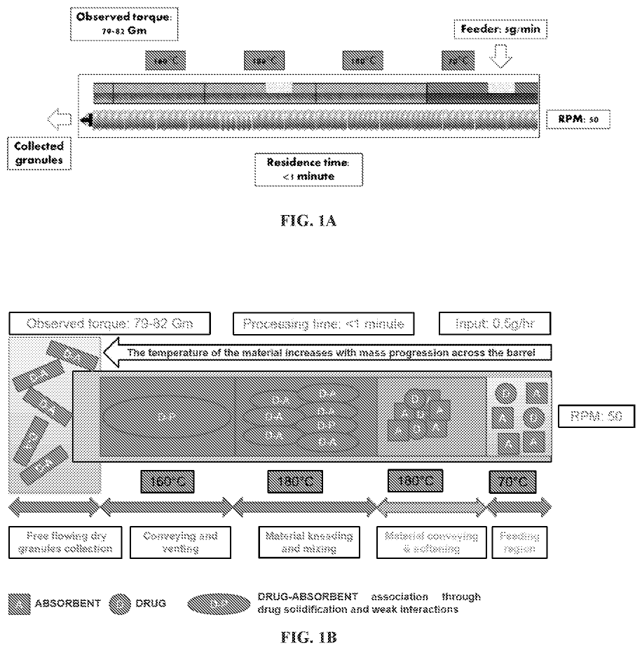

The formulation was prepared by physically mixing indomethacin with Magnesium aluminum silicate, porous silica, and tween 80. The physical mixture was fed into a Leistritz 16 mm nano extruder with four zones. The processing conditions maintained in the four zones are explained in the schematic diagram (See FIG. 1). The produced granules were collected and characterized using various characterization techniques. See FIGS. 2 & 3. Examples B and C are manufactured using the same methods as Example A.

### Example B

TABLE 2
Formulation Composition
Formulation 2

| Ingredient | Purpose | % weight |
|---|---|---|
| Indomethacin | API | 20 |
| Magnesium aluminum silicate | Absorbent | 37.5 |
| Porous silica | Absorbent | 37.5 |
| Tween 80 ® | Surfactant | 5 |

### Example C

TABLE 3
Formulation Composition
Formulation 3

| Ingredient | Purpose | % weight |
|---|---|---|
| Indomethacin | API | 60 |
| Magnesium aluminum silicate | Absorbent | 17.5 |
| Porous silica | Absorbent | 17.5 |
| Tween 80 ® | Surfactant | 5 |

### Example D

TABLE 4
Formulation Composition
Formulation 4

| Ingredient | Purpose | % weight |
|---|---|---|
| Mefenamic acid | API | 20 |
| Magnesium aluminum silicate | Absorbent | 37.5 |
| Silicon dioxide | Absorbent | 37.5 |
| Tween 80 ® | Surfactant | 5 |

### Example E

TABLE 5
Formulation Composition
Formulation 5

| Ingredient | Purpose | % weight |
|---|---|---|
| Mefenamic acid | API | 40 |
| Magnesium aluminum silicate | Absorbent | 27.5 |
| Silicon dioxide | Absorbent | 27.5 |
| Tween 80 ® | Surfactant | 5 |

### Example F

TABLE 6
Formulation Composition
Formulation 6

| Ingredient | Purpose | % weight |
|---|---|---|
| Mefenamic acid | API | 60 |
| Magnesium aluminum silicate | Absorbent | 17.5 |
| Silicon dioxide | Absorbent | 17.5 |
| Tween 80 ® | Surfactant | 5 |

### Example G

TABLE 7
Formulation Composition
Formulation 7

| Ingredient | Purpose | % weight |
|---|---|---|
| Mefenamic acid | API | 80 |
| Magnesium aluminum silicate | Absorbent | 7.5 |
| Silicon diocide | Absorbent | 7.5 |
| Tween 80 ® | Surfactant | 5 |

### Example H

Granules manufactured from Examples A-G were used as raw materials for the manufacturing of solid dosage forms using additive manufacturing such as selective laser sintering.

TABLE 4
Printing Parameters

| Surface temperature (° C.) | 100 | |
|---|---|---|
| Chamber temperature (° C.) | 80 | |
| Kit laser speed | 25 | mm/s |
| Hatching offset | 120 | μm |
| Perimeter offset | 200 | μm |
| Hatching spacing | 25 | μm |
| Layer height | 100 | μm |

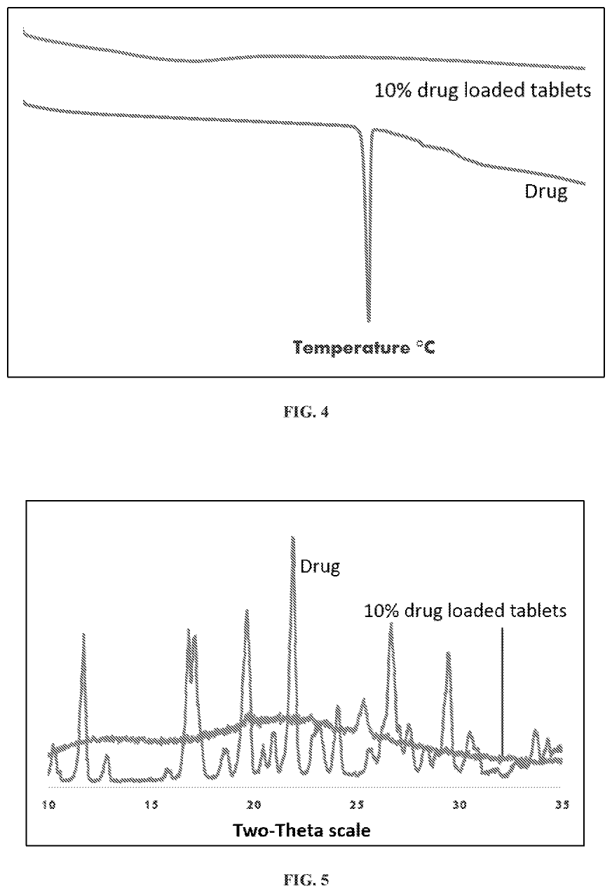

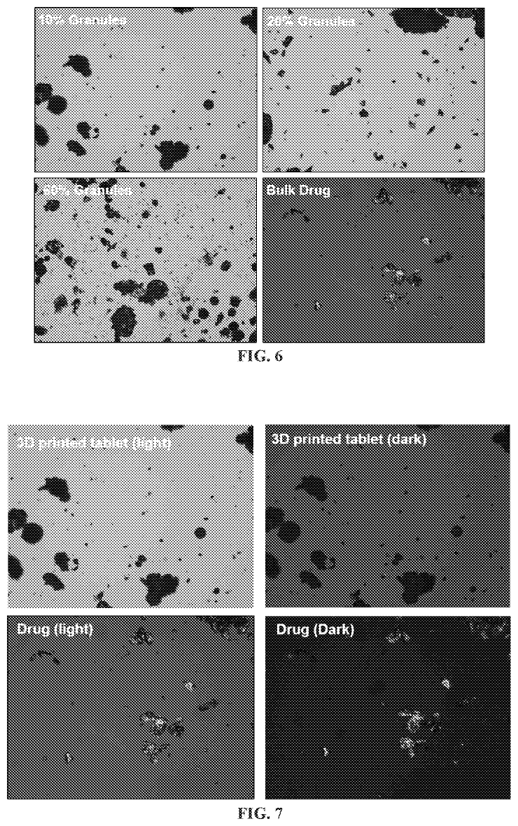

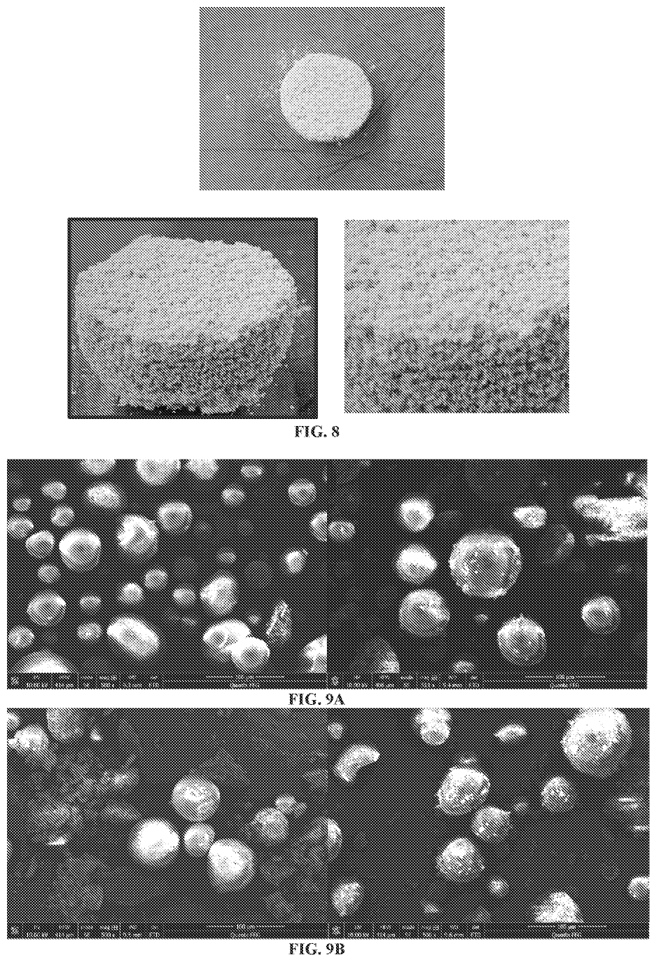

The printed tablets were further characterized as used in FIGS. 4-7. SEM images of compositions Examples D-G are shown FIGS. 9A-9D comparing the unprocessed compositions to the processed compositions.

### Example 2—Characterization of Dosage Form Prepared Via Selective Laser Sintering

### I. Granulation and In-Line Monitoring

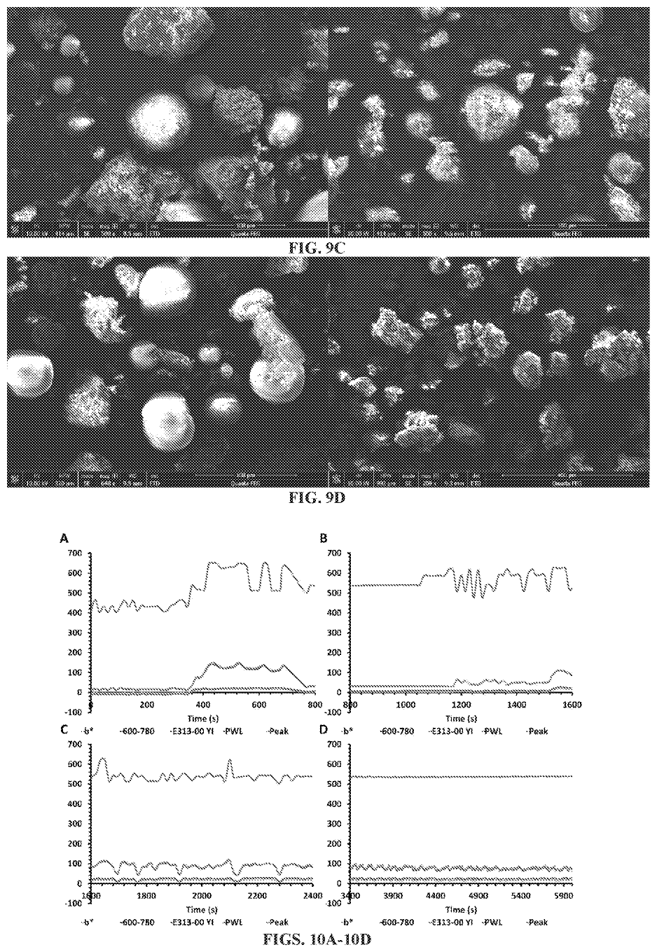

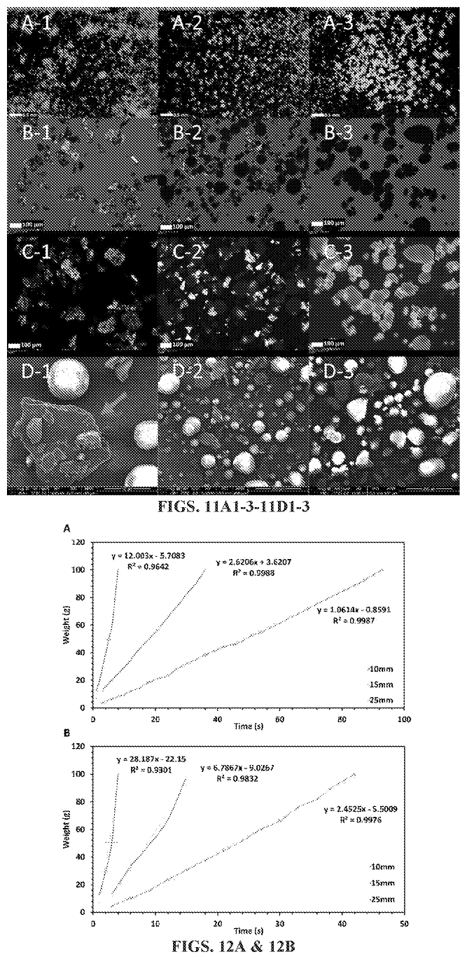

A hot-melt extrusion-based granulation process was developed using a modified screw design with mixing zones throughout the barrel to induce shear mediated melting, mixing, and absorption of the drug on to the inorganic excipients, and the process was monitored using a UV-Visible reflectance probe placed in the ‘zone 6’ of the barrel where the drug was expected to have completely converted into its amorphous form under the right conditions. The granulation process was monitored using the process analytical tool (PAT) to monitor the stability of the process and to monitor the amorphous conversion of IND. CIELAB yellow-blue color space coordinate (b*), and yellowness index (E313-00 YI) were monitored to exploit indomethacin's unique property as in its crystalline γ-form it exhibits a pinkish white appearance, whereas on amorphous conversion its color changes to yellow (Tanabe et al., 2012). Fluctuations in these above-mentioned parameters were expected to indicate a combination of crystalline and amorphous indomethacin in zone 6. These fluctuations can be observed in FIGS. 10A-10C which depict incomplete amorphous conversion of indomethacin which was also observed in the pXRD patterns for the collected samples at these temperatures, 140-150° C. The other monitored parameters such as the custom selected wavelength (600-700 nm) which depicts the average response between 600 and 700 nm, wavelength of maximum reflectance over the measured region (PWL), and reflectance value at the PWL (Peak) were utilized to monitor the stability of process as the fluctuations in these parameters were expected to be a result of improper mixing which would eventually lead to a content uniformity with a broader standard deviation. All the monitored parameters stabilized at 155° C. as seen in FIG. 10D. The samples collected at 155° C. after the process was stabilized were found to be amorphous on pXRD analysis hence the batches for SLS 3D printing were manufactured at 155° C. and the process was monitored using the above mentioned PAT and parameters.

Moreover, the drug content uniformity conducted by sampling the PM-I from three different regions for the three manufactured batches was found to be 101.17±5.64%, 103.68±7.64%, and 100.25±6.67% respectively, whereas the granules collected after HME processing were found to have content uniformity of 100.39±0.51%, 99.86±0.93%, 99.87±0.85% respectively.

### II. Bulk Properties Testing

Previous research has shown that indomethacin has extremely poor flow properties by the means of Angle of Repose (AOR) and Hausner's ratio (Semjonov et al., 2018).

On inspecting indomethacin drug crystals using digital microscopy (FIG. 11A-1), polarized light microscopy (FIGS. 11B-1 & 11C-1), and scanning electron microscopy (FIG. 11D-1) it was observed that indomethacin crystal have uneven and rough surfaces which highly contribute to the observed poor flow properties.

The AOR analysis of IND was conducted as a reference to the man-ufactured granules, and it was found to have a 0 value of 58.2±0.06°, which is classified as ‘very poor’ as per the AOR reference table. Furthermore, the flow through orifice analysis of pure IND could not be conducted as it clogged the orifice and was observed to have no flow throughout the test.

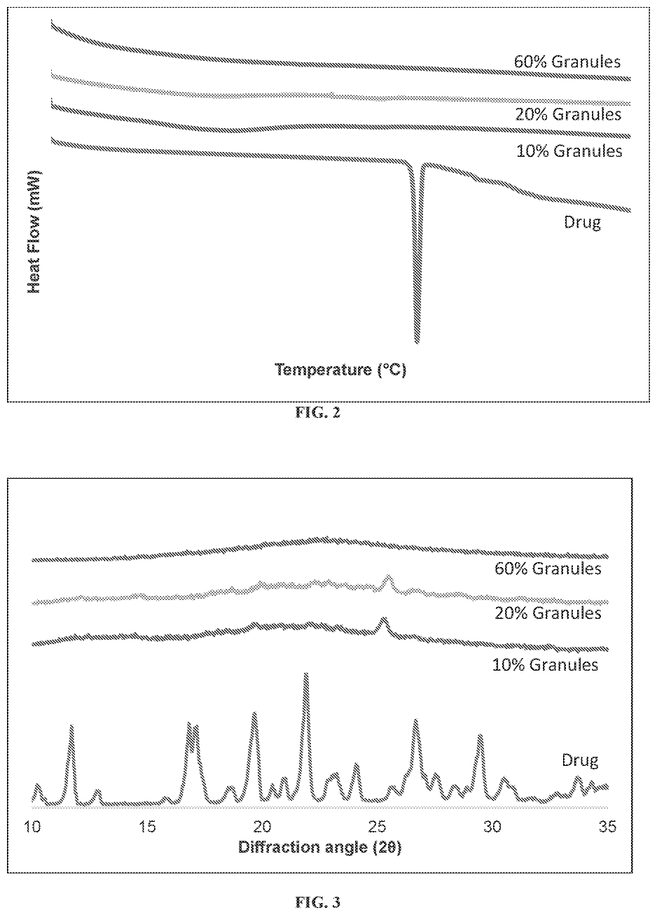

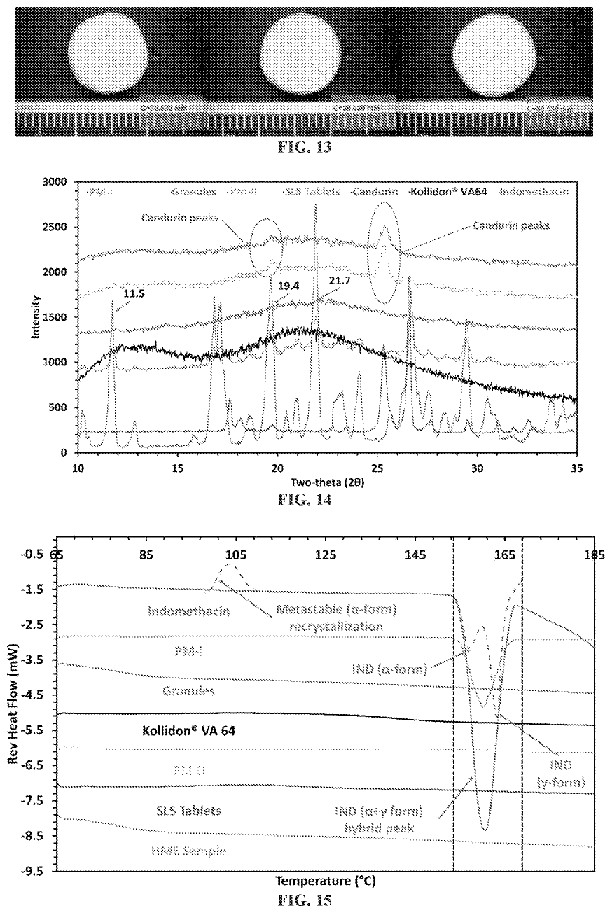

Moving forward to the physical mixtures depicted in FIGS. 11A-2 & 11D-2 which show the discrepancies between the sizes of the drug crystals and the inorganic excipients further shed light on the broad standard deviations observed for PM-I. Physical mixtures having components with different densities tend to segregate when exposed to prolonged vibrations during mass transfer processes. PM-I depicted irregular trends because of repetitive clogging of the orifice and non-reproducible results when exposed to flow through orifice analysis and hence these trends were not depicted in the results section. Further, FIGS. 11B-2 & 11C-2 depict the PLM images of PM-I under the microscope which highlight the presence of crystallinity along with other amorphous inorganic excipients in the blend which do not show any birefringence and hence are seen as dark spots in the images. The granulation technique was designed to break down the crystalline drug and further facilitate the adsorption of the drug onto the surface of the inorganic excipient. During post-processing, it was observed that the drug crystals completely disappeared as seen in FIGS. 11A-3, 11B-3, 11C-3, & 11D-3. Moreover, the birefringence observed in FIGS. 11B-2 & 11C-2 were not present post-processing in FIGS. 11B-3 & 11C-3 indicating complete amorphous conversion of the drug from its crystalline counterpart. It can also be seen in FIGS. 11A-3 & 11D-3 that the granules have a smooth and round surface which can translate to better flow properties as compared to PM-I. The AOR of the extruded granules were observed to have a θ value of 29.49 1.24 degrees, which is classified as ‘excellent’. On conducting the flow through orifice test for the granules and PA-12 (SLS reference material), it was observed that the granules depicted a reproducible trend just like PA-12 with an R² value of ‘>0.9’ for all three orifices (10, 15, 25 mm). Although both the materials demonstrated different weight/minute values, the rationale of this test was to stimulate the flow in a hopper or other mass transfer situations and inspect if the flow follows a reproducible trend. The granules observed excellent trends and no significant deviation as seen in FIG. 12.

### III. 3D Printing Process

During the SLS 3D printing process, 100 μm thick layers of the PM-II from the feed region were spread onto the build platform where the particles were sintered together utilizing a laser as per the design of the tablets. During the build cycle, the build surface maintained its smooth texture as the PM-II exhibited excellent flow properties as expected and there were no print failures or defects experienced during the printing process after the processing parameters were fixed. The twelve tablets manufactured per build cycle were isolated from the build chamber and de-dusted to remove the part cake using a nozzle-based air dispenser. The tablet dimensions were measured and are depicted in Table 1. It was observed that all the tablets were printed as per the dimensions of the designed tablet with an insignificant standard deviation and batch to batch variation, moreover, the deviation in the weights was within the defined range as per the United States Pharmacopoeia (USP).

TABLE 1
Quality control characteristics of the manufactured 3D printed tablets.

| Tablet | Height | Diameter | Weight | Volume | Density | Hardness | Disintegration |
|---|---|---|---|---|---|---|---|
| batch no. | (mm) | (mm) | (mg) | (mm³) | (kg/m³) | (kp) | time (s) |
| I | 5.06 ± 0.03 | 11.98 ± 0.03 | 301 ± 4.24 | 570.55 ± 3.86 | 527.54 ± 3.86 | 8.44 ± 0.88 | 57 ± 7 |
| II | 5.05 ± 0.04 | 11.99 ± 0.01 | 299 ± 5.65 | 570.93 ± 3.32 | 523.67 ± 6.86 | 8.06 ± 1.29 | 42 ± 13 |
| III | 5.07 ± 0.02 | 12.00 ± 0.01 | 303 ± 4.24 | 573.68 ± 3.75 | 528.15 ± 3.94 | 8.26 ± 1.40 | 58 ± 11 |
| Average | 5.06 | 11.99 | 301.00 | 571.72 | 526.45 | 8.25 | 52 |
| S.D. | 0.01 | 0.01 | 2.00 | 1.71 | 2.43 | 0.19 | 7 |
| RSD (%) | 0.20 | 0.08 | 0.66 | 0.30 | 0.46 | 2.30 | 13.46 |

The target weight for these tablets was 300 mg and as per USP 7.5% weight variation is acceptable for tablets with the mentioned weight, therefore a deviation of ±22.5 mg would be considered acceptable, although the observed deviation was extremely narrow. Furthermore, the drug content for each manufactured batch (12 tablets per batch) was estimated (n=2). Batch 1 observed a drug content of 29.4±1.6 mg, Batch 2 observed a drug content of 28.9±1.8 and Batch 3 observed a drug content of 30.2±0.9, the drug release (%) calculations were based on these average values of each batch.

The volume and the density were calculated from the dimensions and the weight of the tablets, the density of a single layer can be used to predict the dimensions for a tablet with the desirable weight. The hardness of the tablets was also found to be reproducible. The inherent property of SLS 3D printing allows the manufacturing of porous tablets (FIG. 13). Even though the tablets have sufficient hardness, they disintegrate in less than 60 s when they come in contact with aqueous buffers.

### IV. Solid-State Characterization

X-ray diffraction has found applicability as one of the most reliable and straightforward techniques for qualitative and quantitative analysis of crystallinity. One of the major aims of this research was the complete amorphous conversion of the drug post HME processing and SLS 3D printing. XRD was used as a tool to optimize the manufacturing temperature and to inspect the solid-state of the optimized granules and manufactured tablets. For the pure drug, all the characteristic peaks of IND (γ-form) were observed (FIG. 14) at 11.5, 19.4, and 21.7 two-theta (20) degrees, which comply with previous reports (Otsuka et al., 2000).

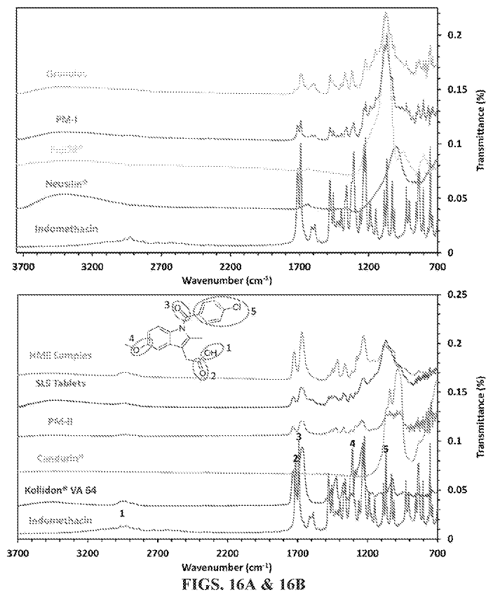

Kollidon® VA64 is amorphous and hence does not depict any characteristic crystalline peaks on pXRD analysis. Although the trace crystallinity of the polymers can be observed using techniques such as wide-angle X-ray scattering (WAXS), this technique was not used here to focus on the solid-state of the drug and not the polymer. All the characteristic peaks of IND were observed in the PM-I before it was processed using twin-screw extrusion. Post-processing all the peaks disappeared which indicates a change in the solid-state of the drug from its crystalline to its amorphous form. Further, there are two small peaks at 19.8 and 25.2 two-theta (20) degrees, out of which one overlaps with the characteristic peak of IND at 19.4 two-theta (20) de-grees. These peaks belong to Candurin® which is the photo-absorbing species required for the successful sintering of PM-II and hence should not be confused with the presence of trace crystallinity in the SLS 3D printed tablets (Davis et al., 2020). This can further be proven by the mDSC results depicted in FIG. 15. The thermal analysis of all the samples was conducted to verify the absence of crystallinity in the granules and the SLS 3D printed tablets, as the drug in its amorphous form does not exhibit a melting endotherm. The melting endotherm of IND was observed in both the pure drug and the PM-I sample which represents the melting point (Tₘ=160.4° C.) of IND (for the γ-form). Moreover, mDSC also revealed the presence of polymorphism in the IND drug crystals. Indomethacin has a glass transition (T_{g}) of 42° C., after which it recrystallizes to its metastable α-form (FIG. 15) close to 100° C. which shows a melting endotherm at 152-154° C. as seen in FIG. 15, whereas the more stable γ form melts at 160° C. on the application of heat energy, this transition has been depicted by the means of dotted lines in FIG. 15 (Abiad et al., 2009). Observations made from the pXRD data were further bolstered by the DSC data where no melting endotherms were observed for the granules or the 3D printed tablets. These results suggest that the solid-state of the drug was amorphous in the granules and tablets. Furthermore, it was also observed that IND starts to degrade on increasing the temperature over its Tₘ. It has been previously observed that IND starts to degrade over its Tₘ when exposed to the conditions for an extended amount of time and gets converted to decarboxylated IND (Shimada et al., 2018).

Hence, the HME conditions selected (155° C.) were safe for processing IND as it was below the Tₘ and the degradation temperature (Td) of the drug. Further FT-IR studies conducted on all the samples provided further insight into the solid-state as well as the interactions between IND and other ingredients in the formulation. FT-IR samples of IND had confirmatory peaks at 2926.6 cm⁻¹ (O—H stretching vibration), 1717.0 cm⁻¹ (C—O stretching of carboxylic acid dimer), 1691.5 cm⁻¹ (C—O stretching of benzoyl group), 1307.7 cm⁻¹ (C—O), and 1068.0 cm⁻¹ (C—Cl) which are represented in FIG. 16.

The carbonyl groups part of amides (associated with nitrogen atoms) usually exhibits peaks at lower wavenumbers (1640 cm⁻¹)(Garbacz and Wesolowski, 2018). Although, the group being an indole ketone, experiences a reduction in the contribution of the mesomeric effect (where nitrogen can donate its lone pair of electrons) in molecules as the nitrogen atom is part of the ring. The mesomerism is responsible for the lower wavenumbers observed for the typical amides and since the effect is absent in IND, the wavenumber for the benzoyl group in the γ-form is relatively higher unhindered groups of carboxylic acid) (Taylor and Zografi, 1997). The formation and exis-tence of these dimers are crucial for the stability and re-crystallization of IND post amorphous conversion. The retention of Peaks 2 and 3 in all samples suggest that there was no chemical degradation or unwanted chemical reaction between IND and other excipients. The changes in the peaks post-processing were expected due to the amorphous conversion of the drug. Post amorphous conversion the benzoyl carbonyl peak shifted from 1691 cm⁻¹ to 1684 cm⁻¹ as in the crystalline γ form. The carbonyl group is stabilized by the hydrogen bonds from the hydroxyl group of the neighboring IND molecule. The peak for the carboxylic acid dimer shifted from 1717.0 cm⁻¹ to 1733.0 cm⁻¹.

As per previous reports post amorphous conversion, IND exists in two forms i.e. cyclic dimers (1706 cm⁻¹) and non-hydrogen bonded carboxylic acid (1735 cm⁻¹) (Ewing el al., 2014). In this study, post-processing IND was observed to dominantly exist in just one state i.e. non-hydrogen bonded carboxylic acid, which might be because of the surfactant and the inorganic excipients as well as their contributions in stabilizing IND in its amorphous form. The absence of dimers might significantly reduce the rate of re-crystallization for the granules although the solid-state stability studies were beyond the scope of this current research and are being conducted as an extension to it. The FT-IR of the 3D printed tablets and the HME samples did not depict strong drug peaks for two reasons, firstly the presence of Kollidon® VA64 overshadowed the drug peaks, and secondly, the drug and the polymer post-processing were expected to form an amorphous solid dispersion. Although there was a resemblance between the FT-IR of the HME and the SLS 3D printed samples which can be attributed to their similar composition and intermolecular interactions.

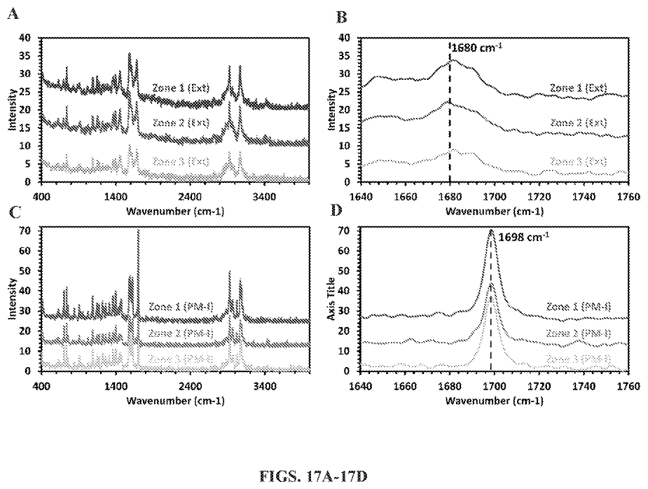

Raman spectroscopy like FT-IR, demonstrated the peak shift from 1700 cm⁻¹ to 1680 cm⁻¹ which represents the benzoyl carbonyl stretching and hence does not give any information of the molecular associations however, it does provide some information on the influence by steric hindrance and tension of molecules imposed by hydrogen-bonded associations in dimmers in the γ form (Garbacz and Wesolowski, 2018; Hédoux, et al., 2009). The sharpness and intensity of this band are associated with the long-range of the dimer chains and this is apparent in the Raman spectra of PM-I (FIG. 17B), in contrast, the peak reduces in intensity and sharpness in the processed granules (FIG. 17A) where the drug is in its amorphous form. Further more, the Raman spectra of IND in its amorphous state are expected to exhibit a broad band around 1679 cm⁻¹ (1698 cm⁻¹ in its crystalline form, FIG. 17D) which corresponds to the dimer associations discussed previously. Although the carbonyl peak from the carboxylic acid disappeared from 1698 cm⁻¹ as seen in FIG. 17C, it appears as a weak broad peak at 1680 cm⁻¹ which confirms that the dimers do not exist in excessive quantities in the granules. This peak shift is prominent, and the intensity is quantifiable when IND is converted to its amorphous form by other techniques such as quench cooling (Taylor and Zhang, 1997).

The absence of crystallinity in the HME and SLS formulations following the above-mentioned solid-state characterizations suggests the formation of an amorphous solid dispersion (ASD). Such a transformation was also observed in our previous study with ritonavir and Kollidon® VA 64 (Davis et al., 2020). Post-processing transformation into an amor-phous solid dispersion can prevent recrystallization of the drug on storage and also improve the kinetic solubility of IND. To investigate the solubility advantage post-processing in vitro release testing was performed.

### V. Performance Testing

The in vitro release testing was performed using a pH shift protocol to simulate physiological conditions and contrary to the USP dissolution medium i.e., pH 7.2 phosphate buffer. The performance test was conducted in 750 mL HCl-KCl buffer (pH 2.0) and then in 900 mL phosphate buffer (pH 6.8). The rationale behind the pH shift dissolution study apart from mimicking the physiological condition was the pH-dependent solubility of IND. The drug is weakly acidic (because of the carboxylic group, pKₐ 4.5) and poorly soluble (because of the hydrophobic indole group) in nature (O'Brien et al., 1984). IND is practically insoluble in acidic pH and has a solubility of 7-9 μg/mL at 35° C. in water, although it has a higher solubility in alkaline pH because of ionization of the molecule (Zeleňák et al., 2018). To understand the impact of the granulation process, and further the SLS 3D printing on the dissolution of the drug, it was important to conduct a pH shift dissolution test. Moreover, because of its pKₐ and hydrophobicity, and the amorphous nature of the drug in the samples of interest, it was important to expose it to acidic conditions before exposing it to relatively basic conditions. Weakly acidic and hydrophobic drugs like IND tend to undergo a solid-state transfer from their more reactive and less stable amorphous forms (higher chemical potential) to less reactive and more stable crystalline form (lower chemical potential) (Dubbini et al., 2014; Jermain et al., 2020; Skrdla et al., 2020). If the drug exists in its amorphous form in the formulation, it is crucial to study its stability in the acidic pH as in the physiological environment the formulation will be exposed to it first. Even if the formulation exhibits excellent dissolution at a relatively basic pH in vitro, but is unstable in acidic pH, there is a chance that it might not demonstrate a similar trend and be as effective as predicted due to recrystallization in vivo.

The amorphous form of the drug is of importance here as thermodynamically it has a higher chemical potential as well as reactivity and hence exhibits better and faster dissolution as compared to its stable crystalline counterpart which is less reactive and has a lower chemical potential due to the stability induced by its neighboring molecules (Huang et al., 2016). This phenomenon is even stronger in this case where the IND molecules are stabilized by strong dimers which makes it difficult for the water molecules to break these bonds.

In the case of amorphous solid dispersions, hydrophilic polymers are used to break these intermolecular bonds between drug molecules which are originally in their crystalline form and forms new intermolecular interactions with the polymers or other excipients such as stabilizers and surfactants to prevent recrystallization (Maniruzzaman et al., 2013; Nie et al., 2015) a similar trend was observed in this case as well. For this formulation, the drug was absorbed onto inorganic carriers and was stabilized using polysorbate 80, moreover, the drug was found to be in its amorphous form. When exposed to an acidic environment (FIG. 18B), the pure drug crystals did not show any improvement in their solubility which was expected because of the above-discussed reasons. When the PM-I was exposed to pH 2, it demonstrated a slight improvement over the pure drug which is not significantly different from the pure drug, this slight improvement can be attributed to the presence of polysorbate 80 in the formulation which acts as a surfactant and facilitates the interactions between the drug and the medium by reducing the surface tension. Since the drug in PM-I was still in its crystalline form, the rate of dissolution was extremely slow. The performance of the granules on the other hand is significantly faster than that of the pure drug and the PM-I, although there seems to be a drop in IND solubility around 90-minutes into the dissolution studies which might be due to the recrystallization or precipitation of the dissolved drug. For this study, 0.2 μm filters were used to make sure only the solubilized IND is estimated, and any recrystallized nuclei are filtered out. Moreover, the dissolution was faster than recrystallization and hence the drug concentration increased again on the 120-minute time point. The dissolution rate PM-II was observed to be similar to that of the granules. Furthermore, the SLS printed tablets and the HME samples demonstrated the highest increase in the rate of solubilization as compared to the pure crystalline drug and the PM-I. The dissolution pattern of the HME samples and the tablets were also iden-tical with little variability as both demonstrated around 4% drug release over a 2-hour dissolution test.

When the pH of the dissolution medium was shifted from 2 to 6.8, the granules, PM-II, SLS 3D printed tablets and the HME samples demonstrated >80% drug release in <5 min. This rapid dissolution and completeness of the release of these formulations signified minimum to no recrystallization in the acidic pH although possible nucleation might have taken place in the acidic pH. It can be seen that even though the dissolution rate of the pure drug and PM-I is faster as compared to their dissolution in the acidic pH, it is significantly slower than the other samples. The pure drug and the PM-1 released only 60% of the drug over 2 h. The drug release of the granules peaked at 91% and then depicted a rapid reduction in the drug concentration. This rapid reduction can be attributed to the recrystallization of the solubilized IND in the medium which was expected because even though the drug existed in its amorphous state in the granules which was responsible for its rapid dissolution, there was nothing in the formulation to maintain the drug in its solubilized state (less stable) and hence it recrystallized (more stable) in the medium. This can further be attributed to the nucleation of the drug in the acidic pH which has previously contributed to severe and rapid increase recrystallization in aqueous solutions (Jermain et al., 2020; Taylor and Zhang, 2016; Van Eerdenbrugh et al., 2010). The spike in the standard deviation for granules right after the pH shift was due to an outlier in Batch 2, moreover, the pH shift led to a rapid increase in dissolution of the suspended drug in the solution which was partly responsible for the spike in the standard deviation values. It should be noted that the content uniformity of the granules was within the range as reported in section 3.1. PM-II on the other hand peaked at 88% drug release and then started to recrystallize in the solution, although the recrystallization rate was slightly slower as compared to the granules which can be attributed to the presence of Kollidon® VA 64 in the formulation. The dissolution profile of the SLS printed tablets and the HME samples were still similar, where the HME samples peaked at 92% drug release (30 min after the pH shift) whereas the SLS 3D printed tablets peaked at 100% drug release (5 min after the pH shift). This can be attributed to the formation of IND-Kollidon®VA64 ASDs after processing the physical mixture using SLS 3D printing. Furthermore, the HME samples maintained the saturation of the drug in the medium throughout the dissolution study and the incomplete release can be attributed to the 2-hour exposure of the sample to the acidic pH and recrystallization of the free amorphous drug. Whereas, the SLS 3D printed tablet demonstrated a 100% release within 5 min and a steep decline thereafter to 90% which can be attributed to the recrystallization of solubilized unstable IND in the medium. After the steep decline, the drug concentration for the tablets was maintained above 80% throughout the dissolution study which suggests ASD formation and stabilization by Kollidon® VA64 during the SLS 3D printing process. For the physical mixtures, the polymer dissolves rapidly and does not prevent recrystallization, whereas HME and SLS 3D printed samples are amorphous solid dispersions, which are supersaturating delivery systems where the polymer stabilizes the drug in the system and maintains supersaturation through intermolecular interactions.

### VI. Material and Methods

a. Materials

Indomethacin (Tokyo Chemical Industries. Lot no. D3NIJJR), magnesium aluminometasilicate (Neusilin US 2, Lot no. 901002, Fuji chemical industries co., ltd Toyama pref., Japan), silicon dioxide (Fuji-sil™, Lot no. 906003, Fuji chemical industries co., ltd Toyama pref., Japan), polysorbate 80 (Lot no. BCCB4768, Sigma-Aldrich®, Missouri, USA), vinyl pyrrolidone-vinyl acetate copolymer (Kollidon® VA 64 (average molecular weight 65,000 g/mol), Lot no. 94189624U0, BASF, Ludwigshafen, Germany), potassium aluminum silicate-based pearles-cent pigment (Candurin®, Lot no. W150645X08, Merck KGaA, Darm-stadt, Germany), HPLC grade acetonitrile was purchased from Fisher Scientific (Pittsburgh. Pa.); all other chemicals and reagents were ACS grade or higher.

b. HME Based Granulation Process

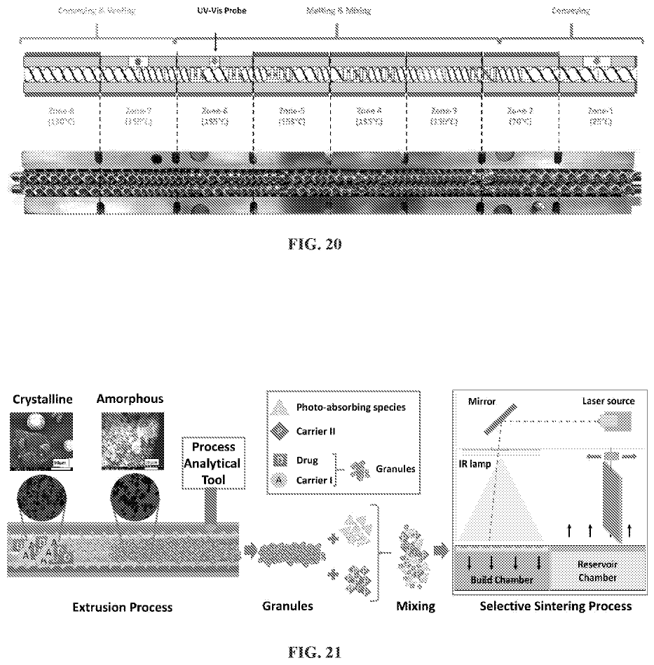

To ensure the reproducibility of the process, three batches of the physical mixture were prepared using the geometric dilution technique. Each 200 g batch of the physical mixture contained a 40% IND drug load, 27.5% of each of the inorganic highly porous absorbents (silicon dioxide and magnesium aluminometasilicate), and 5% of polysorbate 80 (non-ionic surfactant) (here on out this composition will be referred as PM-I). This mixture was transferred to a twin-screw gravimetric feeder with stirring agitators (Brabender Technologie, Ontario, Canada) which was calibrated for the blend to quantify and control the amount of feed going into the system, post-calibration the feed rate was set to 5 g/min. The feed was processed using a twin-screw extruder with a 12 mm outer diameter (OD) (ZSE 12 HP-PH, Leistritz Advanced Technologies Corp., Nuremberg. Germany). The temperature for each zone has been outlined in FIG. 20 along with other processing parameters required to define the process. The granules were collected after the process was stabilized. The physical mixture and the collected granules were subjected to bulk property testing, a series of solid-state characterizations, and performance testing before using them for SLS 3D printing.

c. In-Line Process Monitoring

The HME processing parameters were optimized by using a UV-vis reflectance probe with a 316L Stainless Steel/Nickel alloy tip and sapphire window. The probe was later used as a process analytical tool (PAT) for monitoring the uniformity and amorphous conversion of the subsequent batches (Equitech Int'l Corporation, New Jersey, USA). Indomethacin has a unique property, in its crystalline γ-form, it exhibits a pinkish white appearance, whereas on amorphous conversion its color changes to yellow (Tanabe et al., 2012). During the granulation process, CIELAB yellow-blue color space coordinate (b*), custom selected wavelength (600-700 nm), yellowness index (‘E313-00 YI’ which is supposed to trend with b*), the wavelength of maximum reflectance over the measured region (PWL), and reflectance value at the PWL (Peak) were observed by the reflectance probe and were used as an indicator of amorphous conversion and inspect the stability of the process. The physical mixture was processed with the probe in place with different temperature conditions ranging from 140 to 155° C. (below indomethacin's melting point), the samples were collected, and the yellowness values attained from the probe were noted. These samples were tested using powder X-ray diffraction (pXRD) analysis. Processing conditions where the samples observed no crystalline peaks were selected and the corresponding yellowness values were used to observe the uniformity of subsequent processes.

d. Bulk Properties Testing

i. Digital Light Microscopy

Digital microscopy was used to investigate the morphology of the drug crystals, PM-I, and manufactured granules (Dino light, Torrance, Calif. USA). The microscope was set to a magnification of 65× which was sufficient to observe the particle characteristics of samples. This technique was used as a convenient quality control tool to observe the absence of any independent drug crystals in the manufactured granules post-processing. It was also used to understand the crystal morphology which provided deeper insight into the flow characteristics of the drug. Although digital microscopy was suitable for investigating particle morphology and the presence of independent crystals, it was not suitable to study and observe the inorganic carrier particle surface post-processing. Hence, polarized light microscopy and scanning electron microscopy were conducted for all the above-mentioned samples to further investigate their surface properties and crystallinity of the drug in the samples.

ii. Polarized Light Microscopy (PLM)

Polarized light microscopy using an Olympus BX53 polarizing photomicroscope (Olympus America Inc., Webster, Tx., USA) was used to investigate the crystallinity of IND. PM-I, and the granules. The microscope had a Bertrand Lens and a 10× objective lens. The samples were evenly dispensed on a glass slide which was later dusted off to remove excess powder and a coverslip was placed onto it. The sample slides were then observed using a 10× magnification lens and an appropriate zone was selected to observe the state of the sample. The magnification was then increased to 20× to further observe the crystals with more clarity. Crystalline particles possess the property of birefringence, which is characteristic of crystalline substances, hence it was predicted that the granules will not depict any birefringence. After focusing on the sample, snapshots were taken with a QICAM Fast 1394 digital camera (QImag-ing, BC, Canada). These images were taken with and without a 530 nm compensator (U-TP530, Olympus® corporation, Shinjuku City, Tokyo, Japan). The snapshots were processed using Linksys 32 Software® (Linkam Scientific Instruments Ltd, Tadworth, UK).

iii. Scanning Electron Microscopy

To understand the surface morphology of the drug crystals, physical mixture, and the processed granules, a scanning electron microscope (Quanta FEG 650 ESEM, FET Company. Hillsboro, Oreg., USA) was used. The samples were first exposed to vacuum gold sputtering (EMS Sputter Coater, Hatfield, Pa., USA) before observing them under the microscope. Microscopic images were captured at an accelerated voltage of 10 KV, emission current of 15μÅ, the working distance of 10 mm, and a spot size of 3. The magnification was varied from 100× to 2000× based on the purpose of the observation.

iv. Powder Flow

A United States Pharmacopoeia (USP) compliant flowability tester, with funnel attachments (BEP2, Copley Scientific Limited, Nottingham, UK) was used for the flow-through orifice study. The purpose of this test was to stimulate the flow in a hopper or other mass transfer situations (Taylor et al., 2000). The funnel was placed 40 mm above the collecting beaker, and the beaker was placed on a measuring scale. The nozzle was fixed onto the funnel and the shutter mechanism was used to prevent any premature flow from the funnel. 100 g of the sample powder was transferred to the funnel and the test was started 30 s after the transfer (this facilitated floccule formation). The weight was recorded for the samples with respect to the time in triplicates (n=3) for each nozzle diameter (10, 15, and 25 mm) and samples (PA 12 (reference), granules, and drug). Time versus weight curves were constructed for the processed granules and the properties were compared with PA 12.

v. Angle of Repose

A 100 mm circular test platform together with a digital height gauge having a range of 0-300 mm and an accuracy of 0.03 mm was used (BEP2, Copley Scientific Limited. Nottingham, UK). The test platform had a protruding outer lip in order to retain a layer of the powder upon which the cone was formed. The surplus powder was collected in a tray below the test platform. The nozzle (10 mm nozzle for the angle of repose) of the funnel was placed 75 mm above the test platform, and the nozzle was secured using the shutter mechanism. 100 g of the sample (drug and granules) were placed in the funnel, and the shutter was moved gently but rapidly to allow the powder to flow. The powder formed a conical on the test platform and started overflowing. The sample was allowed to overflow until the pile height was observed to be constant, this protocol was repeated thrice (n=3). The height of the powder cone was measured using the digital height gauge and the diameter of the cone was 100 (diameter of the platform was 100 mm). Equation 2 was used to calculate the angle of repose.

$$tan θ = \frac{height of cone , mm}{Half of cone base diameter , mm} \qquad 1$$

$$θ = {tan}^{- 1} * \frac{height of cone , mm}{50 mm} \qquad 2$$

e. Selective Laser Sintering 3D Printing

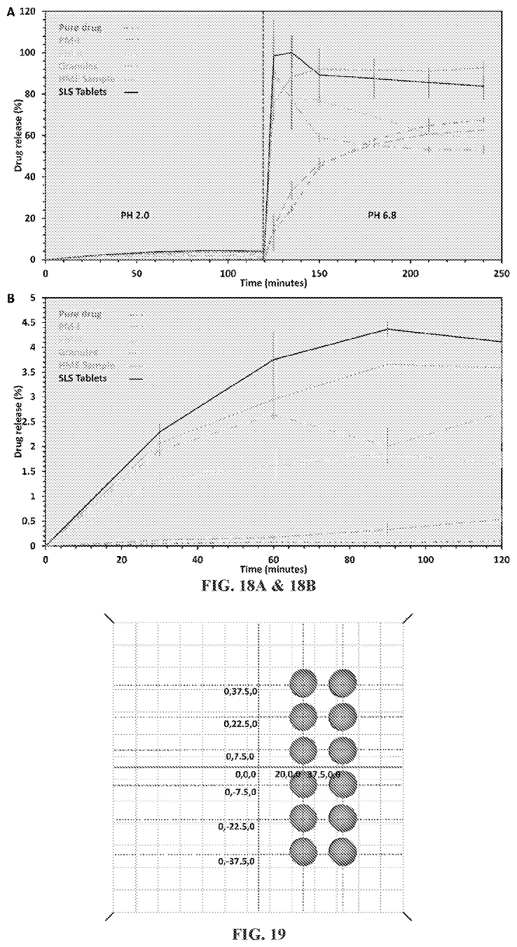

Granules (25% w/w) were mixed with Kollidon® VA 64 (72% w/w), and candurin (3% w/w) (this mixture composition here on out will be referred to as PM-II) by conducting geometric dilutions and using a mortar and pestle and the drug content uniformity was performed post-mixing by withdrawing samples from three distinct regions of the blend. Post blending PM-II was passed through the 12-inch diameter, no. 170 sieve (90 μm pore size) to break down any agglomerates present. The sieve selected had a pore size<100 μm to prevent agglomerates greater than 100 μm which might impact the printing process since the layer thickness set for the process is 100 μm. This powder batch was introduced to the feed region of the benchtop SLS 3D printer (Sintratec kit, Sintratec, Switzerland) equipped with a 2.3 W 455 nm laser. A powder batch of 150 g was used for each build cycle. For the system set up the CAD file with twelve printlet having 5 mm height and 12 mm diameter were loaded onto the Sintratec central software, the coordinates of which have been depicted in FIG. 19. Moving forward the layer height was set to 100 μm, with the number of perimeters set to 1, and the perimeter offset set to 200 μm. The Hatching offset was set to 120 μm, and the hatch spacing was set to 25 μm. After setting up the print parameters, the printing conditions were established where the chamber temperature was set at 80° C. and the surface temperature was maintained at 105° C., which are both below the glass transition point of the polymer (>120° C.) and the melting point of the drug (>160° C.). Chamber temperatures maintained close to or higher than the surface temperature have been observed to form agglomerates and caused fusion of the blend in the feed region which ultimately leads to print failure (Davis el al., 2020). The laser speed was maintained at 50 mm/s. These process parameters were maintained for each build cycle and the build cycle was repeated thrice each time with a virgin powder batch to prevent possible degradation of the drug sub-stance. Each manufacturing lot composed of twelve tablets and all the tablets were tested for their weight, and dimensions using a calibrated VWR® digital caliper (VWR®, PA, U.S.) to evaluate the repeatability of the AM process. Using the observed dimensions of the tablets, their volume was calculated using equation (3) where ‘r’ is the radius and ‘h’ is the height of the tablets. The density was then calculated using the volume and the weight of the tablets using equation (4). Moreover, the tablets from each printed batch were tested for hardness (n=3) using a texture analyzer (TA-XT2 analyzer, Texture Technologies Corp. New York, USA) along with a one-inch cylinder probe apparatus. Briefly, the test speed was set to 0.3 mm/s and the samples were positioned between the probe and base across their diameter. The dimensions of the samples were inserted in the software before the test and the stopping distance for the probe was set to 3 mm from the starting point of the test which was deemed sufficient to assess the hardness of the samples. The first point of drop-in force (peak force) was recorded as the hardness of the samples.

$$Volume ( V ) = {π r}^{2} h \qquad 3$$

$$Density ( ρ ) = \frac{mass}{volume} \qquad 4$$

Furthermore, The disintegration time (n=3) of the samples were assessed using a basket-rack assembly filled with 900 mL pH 2 HCl-KCl buffer and maintained at 37±2° C. in a 1000 mL vessel. Briefly, three tablets were placed in the baskets of the oscillating apparatus, operating at a frequency of 29-32 cycles a minute. The timer was started at the beginning of the test and stopped when the tablets were disintegrated completely with no traces of the samples observed in the basket and the time was recorded. The dissolution studies (n=3), and other solid-state characterization techniques were used to investigate the performanceand characteristics of the samples.

The PM-II was also processed through HME to manufacture ASDs which were then used as a reference amorphous solid dispersion (ASD) formulation to compare with the SLS 3D printed tablets. The HME reference was manufactured using PM-II at 165° C. instead of 155° C. employing the same setup as described in FIG. 20.

f. Solid-State Characterization

i. Powder X-Ray Diffraction (pXRD)

To investigate the solid-state of IND, PM-I, granules, Kollidon® VA 64. PM-II, 3D printed tablets, and hot-melt extruded samples pXRD analysis was conducted. 100-150 mg of the samples were dispensed onto the sample cell, the surface was flattened using a glass slide, the excess powder was discarded, and these cells were placed on the sample holders. The samples were then analyzed using a benchtop pXRD in-strument (MiniFlex, Rigaku Corporation, Tokyo, Japan). The measuring conditions were set to a 2θ angle from 5° to 60°, a scan speed of 2°/minute, scan step of 0.02°, where the resultant scan resolution observed was 0.0025. Moreover, the voltage and the current for the analysis were maintained at 45 V and 15 mV, respectively. The data were collected and plotted as a graph of intensity versus 2θ.

ii. Modulated Differential Scanning Calorimetry (mDSC)

To investigate the presence of crystallinity or degradation mDSC analysis of IND, PM-I, granules, Kollidon® VA 64, PM-II, 3D printed tablets, and hot-melt extruded samples were conducted (DSC Q20, TA® instruments, Delaware, USA). The observations from the analysis were also used to determine the temperatures for the SLS-AM process as well as HME processing. Samples weighing 5-15 mg were dispensed in standard aluminum pans (DSC consumables incorporated, Minnesota. USA) using a microbalance (Sartorius 3.6P microbalance. Germany) and sealed using standard aluminum lids. The analysis was conducted from 60° C. to 200° C., where the ramp rate was set to 5° C./minute, and modulation of 1° C. every 60 s. The collected data were analyzed by developing temperature (° C.) versus reverse heat flow (mW) plots.

iii. Fourier Transform Infrared Spectroscopy (FT-IR)

FT-IR provides insight into the post-processing interactions between different functional groups present on the components. FT-IR analysis was used to investigate the changes in the IND spectrum after each process and the interactions between IND and other components (iS50 FT-IR equipped with a SMART OMNI-Sampler, Nicolet, ThermoFisher Scientific, Waltham. Mass. USA). A sample of 20-25 mg of IND, excipients. PM-I, granules, PM-II, SLS printed tablets, and extruded filaments (powdered for the analysis) were dispensed on the sample holder and their % transmittance was measured using a range of 3100-700 cm⁻¹. The resolution of the test was set to 4 cm⁻¹ with 64 scans per run.

To ensure the absence of contamination from previous samples, the cell was cleaned using isopropanol and the background spectrum was collected between each sample. The raw data were translated into spectra which were then investigated for intermolecular interaction, stability, and the solid-state of the samples using OMNIC™ series software (ThermoFisher Scientific, Waltham. Mass., USA).

iv. Raman Mapping

Raman surface mapping was conducted for the pure drug, the physical mixture, as well as the granules to evaluate the changes in the inelastic scattering between the samples and also check the drug distribution on the surface of the sample using an iS™ 50 Raman module (ThermoFisher Scientific, Waltham, Mass., USA) equipped with an Indium Gallium Arsenide (InGaAs) detector, and an XT-KBr beam splitter. The samples were loaded on the sample holder and the sample surface was focused on using the associated microscope and camera, three different zones were analyzed for each sample to investigate the difference in the intensity of their spectrum which could be an indicator of the drug distribution throughout the sample. The power was set to 0.50 W, the spectra were collected from 100 to 4000 cm⁻¹ with a resolution of 4 cm⁻¹, and the number of runs was set to 32 to reduce the noise. The data were collected as shifted spectrum and were plotted against the observed intensity.

g. HPLC Method of Analysis

The HPLC method was adopted from a previously conducted study and further modified for this study (Novakova et al., 2005). IND was estimated using reverse phase-high performance liquid chromatographic (RP-HPLC) analysis (Agilent 1100 series. Agilent Technologies, Santa Clara, Cali-fornia, USA). A 250 mm×4.6 mm, 5 μm particle size, stainless steel C-18 column (Nucleosil®-100-5C18 (Suppleco series), Millipore Sigma, Burlington, Mass.) was used for the analysis. 0.2% o-phosphoric acid with acetonitrile (30:70) was used as the mobile phase (the mobile phase ratio was modified to improve the peak sharpness and sensitivity of the HPLC method). The flow rate and the injection volume were set to 1.2 mL/min and 5 μL, respectively. The retention time (R_{T}) was observed to be 4.8 min and hence the run duration was maintained at 6 min. Indomethacin was detected using a UV-vis detector (Agilent 1100 series, Agilent Technologies. Santa Clara. Calif.) at a wavelength of 237 nm. A calibration curve ranging from 0.5 to 8 μg/mL was used for the quantification of indomethacin (R² 0.999) to assess the reliability and linearity of the method. All standards were prepared in ACN, and samples were diluted using ACN. For the content uniformity and drug content studies, ACN was used to extract IND from the samples before analysis.

h. In Vitro Release Testing (IV-RT)

The performance of the manufactured granules and the 3D printed tablets were tested against pure crystalline IND, PM-I, PM-II, and HME samples using in-vitro release testing. For this pH-shift dissolution test, the above-mentioned samples (n=3) were introduced into 750 mL of HCl-KCl buffer (pH 2, 0.1 M, hydrochloric acid-potassium chloride buffer) for 2 h using a 900 mL vessel. 150 mL of phosphate buffer (pH6.8, 0.1 M) was then introduced to each vessel shifting the pH from 2 to 6.8, with a final volume of 900 mL. It is important to note that the phosphate buffer should be prepared for a final volume of 900 mL, hence the above-mentioned 150 mL volume is the concentrated buffer. The dissolution of the samples was tested at the final pH of 6.8 for an additional 2 h. The test was conducted using a Paddle type assembly (USP type II). The test was conducted using a standard dissolution apparatus (Vankel VK 7000, Agilent Technologies, Santa Clara, Calif., USA) at 37.5° C., and the paddles were maintained at 50 RPM throughout the study. Sample media of 1 mL were drawn using 0.2 μm polyethersulfone filters (VWR International. Radnor, Pa. USA) with an autosampler (Vankel VK 8000, Agilent Technologies, Santa Clara, Calif. USA) at predetermined time points. The sample volume was replaced with 1 mL fresh buffer (HCl-KCU/Phosphate buffer) to maintain the volume in the vessels. Acetonitrile was used to dilute (2-fold) the drawn samples and the previously described method of analysis was used to quantify the API in the samples.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplarv procedural or other details supplementary to those set forth herein, are specifically incorporated herein by reference.
Abiad et al., Food Eng. Rev., 1:105-132, 2009.
Davis et al., J. Pharm. Sci., 2020.
Davis et al. AAPS PharmSciTech, 21:319, 2020.
Dubbini et al., Int. J. Pharma., 473:536-544, 2014.
Ewing et al., Eur. J. Pharm. Sci., 60:64-71, 2014.
Garbacz and Wesolowski, Molecules, 23:2136, 2018.
Hédoux et al., Eur. J. Pharm. Sci., 38:156-164, 2009.
Huang et al., J. Pharm. Sci., 105:3549-3561, 2016.
Jermain et al., Mol. Pharm., 2020.
Maniruzzaman et al., Int. J. Pharm., 443:199-208, 2013.
Nie et al., Mol. Pharm., 12:2459-2468, 2015.
Nováková et al., J. Pharm. Biomed. Anal., 37:899-905.
O'Brien et al., Analytical Profiles ofDrug Substances, pg. 211-238, 1984.
Otsuka et al., AAPS PharmSci., 2:E9, 2000.
Semjonov et al., Int. J. Pharm., 541:188-197, 2018.
Shimada et al., Int. J. Pharm., 545:51-56, 2018.
Skrdla et al., Mol. Pharm., 17:219-228, 2020.
Tanabe et al., Int. J. Pharm., 429:38-45, 2012.
Taylor et al., AAPS PharmaSciTech, 1:20-30, 2000.
Taylor and Zhang, Pharm. Res., 14:1691-1698, 1997.
Taylor and Zhang. Adv. Drug. Deliv., 101:122-142, 2016.
Taylor and Zografi, Pharm. Res., 14:1691-1698, 1997.
Van Eerdenbrugh et al., J. Pharm. Sci., 99:3826-3838, 2010.
Zeleňák et al., Appl. Surf. Sci., 443:525-534, 2018.

What is claimed is:

1. A pharmaceutical composition comprising:
(A) an active pharmaceutical ingredient;
(B) a first absorbent;
(C) a second absorbent; and
(D) a surfactant;
wherein the pharmaceutical composition has a Carr's Index of greater than about 4 and flowability measured by the angle of repose of equal to or less than about 40.

2. The pharmaceutical composition of claim 1, wherein the pharmaceutical composition is present as free-flowing particles.

3. The pharmaceutical composition of claim 1, wherein the pharmaceutical composition present as agglomerates.

4. The pharmaceutical composition according to any one of claims 1-3, wherein the pharmaceutical composition comprises an amorphous active pharmaceutical ingredient.

5. The pharmaceutical composition according to any one of claims 1-3, wherein the pharmaceutical composition comprises a semi-crystalline active pharmaceutical ingredient.

6. The pharmaceutical composition according to any one of claims 1-3, wherein the pharmaceutical composition comprises a crystalline active pharmaceutical ingredient.

7. The pharmaceutical composition according to any one of claims 1-6, wherein the active pharmaceutical ingredient is absorbed on the first absorbent or the second absorbent.

8. The pharmaceutical composition of claim 7, wherein the active pharmaceutical ingredient is absorbed on the first absorbent.

9. The pharmaceutical composition of claim 7, wherein the active pharmaceutical ingredient is absorbed on the second absorbent.

10. The pharmaceutical composition according to any one of claims 7-9, wherein the absorbed active pharmaceutical ingredient causes the first absorbent or the second absorbent to form an agglomeration.

11. The pharmaceutical composition according to any one of claims 1-10, wherein the active pharmaceutical ingredient and the first absorbent are homogenously mixed.

12. The pharmaceutical composition according to any one of claims 1-10, wherein the active pharmaceutical ingredient and the second absorbent are homogenously mixed.

13. The pharmaceutical composition according to any one of claims 1-12, wherein the first absorbent and the second absorbent are homogenously mixed.

14. The pharmaceutical composition according to any one of claims 1-13, wherein the active pharmaceutical ingredient, the first absorbent, and the second absorbent are homogenously mixed.

15. The pharmaceutical composition according to any one of claims 1-14, wherein the active pharmaceutical ingredient is a poorly soluble drug.

16. The pharmaceutical composition according to any one of claims 1-14, wherein the active pharmaceutical ingredient is a BCS class 1 drug.

17. The pharmaceutical composition according to any one of claims 1-15, wherein the active pharmaceutical ingredient is a BCS class 2 drug.

18. The pharmaceutical composition according to any one of claims 1-15, wherein the active pharmaceutical ingredient is a BCS class 3 drug.

19. The pharmaceutical composition according to any one of claims 1-15, wherein the active pharmaceutical ingredient is a BCS class 4 drug.

20. The pharmaceutical composition according to any one of claims 1-19, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintic, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

21. The pharmaceutical composition according to any one of claims 1-20, wherein the pharmaceutical composition comprises from about 5% w/w to about 90% w/w of the active pharmaceutical ingredient.

22. The pharmaceutical composition according to any one of claims 1-21, wherein the pharmaceutical composition comprises from about 10% w/w to about 80% w/w of the active pharmaceutical ingredient.

23. The pharmaceutical composition according to any one of claims 1-22, wherein the pharmaceutical composition comprises from about 20% w/w to about 60% w/w of the active pharmaceutical ingredient.

24. The pharmaceutical composition according to any one of claims 1-22, wherein the pharmaceutical composition comprises from about 10% w/w to about 40% w/w of the active pharmaceutical ingredient.

25. The pharmaceutical composition according to any one of claims 1-22, wherein the pharmaceutical composition comprises from about 40% w/w to about 80% w/w of the active pharmaceutical ingredient.

26. The pharmaceutical composition according to any one of claims 1-25, wherein the first absorbent is a silicate.

27. The pharmaceutical composition of claim 26, wherein the silicate is a silicate salt.

28. The pharmaceutical composition of claim 27, wherein the silicate is an aluminum silicate.

29. The pharmaceutical composition according to any one of claims 26-28, wherein the silicate is magnesium aluminum silicate.

30. The pharmaceutical composition according to any one of claims 1-29, wherein the pharmaceutical composition comprises from about 2.5% w/w to about 45% w/w of the first absorbent.

31. The pharmaceutical composition according to any one of claims 1-30, wherein the pharmaceutical composition comprises from about 5% w/w to about 40% w/w of the first absorbent.

32. The pharmaceutical composition according to any one of claims 1-31, wherein the pharmaceutical composition comprises from about 10% w/w to about 25% w/w of the first absorbent.

33. The pharmaceutical composition according to any one of claims 1-31, wherein the pharmaceutical composition comprises from about 30% w/w to about 40% w/w of the first absorbent.

34. The pharmaceutical composition according to any one of claims 1-33, wherein the second absorbent is silica or aluminum comprising a plurality of pores.

35. The pharmaceutical composition according to any one of claims 1-34, wherein the second absorbent is silica.

36. The pharmaceutical composition according to any one of claims 1-35, wherein the second absorbent is silica comprising a plurality of pores, wherein the pores comprise a diameter between about 0.1 nm and about 50 nm.

37. The pharmaceutical composition of claim 36, wherein the pores have a diameter between 2 nm and about 50 nm.

38. The pharmaceutical composition according to any one of claims 1-37, wherein the pharmaceutical composition comprises from about 2.5% w/w to about 45% w/w of the second absorbent.

39. The pharmaceutical composition according to any one of claims 1-38, wherein the pharmaceutical composition comprises from about 5% w/w to about 40% w/w of the second absorbent.

40. The pharmaceutical composition according to any one of claims 1-39, wherein the pharmaceutical composition comprises from about 10% w/w to about 25% w/w of the second absorbent.

41. The pharmaceutical composition according to any one of claims 1-39, wherein the pharmaceutical composition comprises from about 30% w/w to about 40% w/w of the second absorbent.

42. The pharmaceutical composition according to any one of claims 1-41, wherein the pharmaceutical composition comprises the same amount of the first absorbent and the second absorbent.

43. The pharmaceutical composition according to any one of claims 1-42, wherein the surfactant is a polysorbate derivative.

44. The pharmaceutical composition of claim 43, wherein the surfactant is poly(ethylene glycol) derivatized polysorbate.

45. The pharmaceutical composition of claim 44, wherein the surfactant comprises from about 10 to about 30 poly(ethylene glycol) repeating units.

46. The pharmaceutical composition of claim 45, wherein the surfactant comprises 20 poly(ethylene glycol) repeating unit.

47. The pharmaceutical composition according to any one of claims 43-46, wherein the surfactant comprises a fatty acid.

48. The pharmaceutical composition of claim 47, wherein the fatty acid is oleic acid.

49. The pharmaceutical composition according to any one of claims 1-48, wherein the pharmaceutical composition comprises from about 0.5% w/w to about 20% w/w of the surfactant.

50. The pharmaceutical composition according to any one of claims 1-49, wherein the pharmaceutical composition comprises from about 1% w/w to about 10% w/w of the surfactant.

51. The pharmaceutical composition according to any one of claims 1-50, wherein the pharmaceutical composition comprises from about 2.5% w/w to about 7.5% w/w of the surfactant.

52. The pharmaceutical composition according to any one of claims 1-51, wherein the pharmaceutical composition comprises an excipient.

53. The pharmaceutical composition of claim 52, wherein the excipient is a laser absorbing species.

54. The pharmaceutical composition according to any one of claims 1-53, wherein the pharmaceutical composition comprises a second active pharmaceutical ingredient.

55. The pharmaceutical composition according to any one of claims 1-54, wherein the pharmaceutical composition comprises a pharmaceutically acceptable polymer.

56. The pharmaceutical composition according to any one of claims 1-55, wherein the pharmaceutical composition is substantially free of any other compound.

57. The pharmaceutical composition according to any one of claims 1-56, wherein the pharmaceutical composition is essentially free of any other compound.

58. The pharmaceutical composition according to any one of claims 1-57, wherein the pharmaceutical composition is entirely free of any other compound.

59. The pharmaceutical composition according to any one of claims 1-58, wherein the pharmaceutical composition is substantially free of any other compound other than the active pharmaceutical ingredient, the first absorbent, the second absorbent, an excipient, a second active pharmaceutical ingredient, or a pharmaceutically acceptable polymer.

60. The pharmaceutical composition according to any one of claims 1-59 further comprising subjecting the pharmaceutical composition to milling.

61. The pharmaceutical composition according to any one of claims 1-60 further comprising formulating the pharmaceutical composition into a unit dose.

62. The pharmaceutical composition of claim 61, wherein the unit dose is formulated for oral delivery.

63. The pharmaceutical composition of claim 62, wherein the oral delivery is formulated as a tablet, capsule, or suspension.

64. The pharmaceutical composition according to any one of claims 1-63, wherein the pharmaceutical composition comprises a Carr's Index from about 5 to about 25.

65. The pharmaceutical composition according to any one of claims 1-64, wherein Carr's Index is from about 5 to about 15.

66. The pharmaceutical composition according to any one of claims 1-65, wherein the pharmaceutical composition comprises a surface area of greater than 100 m²/g.

67. The pharmaceutical composition of claim 66, wherein the surface area is greater than 200 m²/g.

68. The pharmaceutical composition of either claim 66 or claim 67, wherein the surface area is from about 100 m²/g to about 500 m²/g.

69. The pharmaceutical composition according to any one of claims 66-68, wherein the surface area is 150 m²/g to about 400 m²/g.

70. The pharmaceutical composition according to any one of claims 1-69, wherein the pharmaceutical composition comprises a mean or average particle size distribution of greater than about 25 μm.

71. The pharmaceutical composition of claim 70, wherein the mean or average particle size distribution is greater than about 50 μm.

72. The pharmaceutical composition of claim 70, wherein the mean or average particle size distribution is from about 25 μm to about 500 μm.

73. The pharmaceutical composition according to any one of claims 70-72, wherein the mean or average particle size distribution is from about 50 μm to about 250 μm.

74. The pharmaceutical composition according to any one of claims 70-73, wherein the mean or average particle size distribution is from about 60 μm to about 100 μm.

75. The pharmaceutical composition according to any one of claims 1-74, wherein the pharmaceutical composition has a flowability as a function of angle of repose of less than about 35.

76. The pharmaceutical composition of claim 75, wherein the flowability is from about 5 to about 35.

77. The pharmaceutical composition according to any one of claims 75-76, wherein the flowability is from about 15 to about 30.

78. The pharmaceutical composition according to any one of claims 75-77, wherein the flowability is from about 25 to about 30.

79. The pharmaceutical composition according to any one of claims 1-78, wherein the pharmaceutical composition comprises a drug content uniformity of greater than about 75%.

80. The pharmaceutical composition of claim 79, wherein the drug content uniformity is greater than 80%.

81. The pharmaceutical composition of either claim 79 or claim 80, wherein the drug content uniformity is from about 90% to about 110%.

82. The pharmaceutical composition according to any one of claims 79-81, wherein the drug content uniformity is from about 95% to about 105%.

83. The pharmaceutical composition according to any one of claims 1-82, wherein the pharmaceutical composition is formulated as granules.

84. The pharmaceutical composition according to any one of claims 1-83, wherein the pharmaceutical composition comprises:
(A) about 20% w/w to about 60% w/w indomethacin;
(B) about 17.5% w/w to about 37.5% w/w magnesium aluminum silicate;
(C) about 17.5% w/w to about 37.5% w/w porous silica; and
(D) about 5%0/w/w of Tween® 80.

85. The pharmaceutical composition according to any one of claims 1-83, wherein the pharmaceutical composition comprises:
(A) about 20% w/w to about 80% w/w mefenamic acid;
(B) about 7.5% w/w to about 37.5% w/w magnesium aluminum silicate;
(C) about 7.5% w/w to about 37.5% w/w porous silica; and
(D) about 5% w/w of Tween® 80.

86. A method of preparing a pharmaceutical composition comprising:
(A) obtaining a mixture of an active pharmaceutical ingredient, a first absorbent, a second absorbent, and a surfactant; and
(B) subjecting the mixture to an extrusion process to obtain a pharmaceutical composition.

87. The method of claim 86, wherein the extrusion process is performed with a hot melt extruder.

88. The method of either claim 86 or claim 87, wherein the extrusion process is performed at a temperature greater than the melting point of the active pharmaceutical ingredient.

89. The method according to any one of claims 86-88, wherein the extrusion process comprises four stages.

90. The method according to any one of claims 86-89, wherein the first stage comprises a first temperature from about 30° C. to about 150° C.

91. The method of claim 90, wherein the first temperature is from about 50° C. to about 100° C.

92. The method according to any one of claims 86-91, wherein the second stage comprises a second temperature from about 75° C. to about 250° C.

93. The method of claim 92, wherein the second temperature is from about 125° C. to about 200° C.

94. The method according to any one of claims 86-93, wherein the third stage comprises a third temperature from about 75° C. to about 250° C.

95. The method of claim 94, wherein the third temperature is from about 125° C. to about 200° C.

96. The method according to any one of claims 86-95, wherein the fourth stage comprises a fourth temperature from about 75° C. to about 250° C.

97. The method of claim 96, wherein the fourth temperature is from about 125° C. to about 200° C.

98. The method according to any one of claims 86-97, wherein the extrusion process comprises a feed rate from about 1 g/min to about 25 g/min.

99. The method of claim 98, wherein the feed rate is from about 2.5 g/min to about 10 g/min.

100. The method according to any one of claims 86-97, wherein the extrusion process comprises a speed from about 10 revolutions per minute (rpm) to about 250 rpm.

101. The method of claim 100, wherein the speed is from about 25 rpm to about 100 rpm.

102. The method of claim 101, wherein the speed is about 50 rpm.

103. The method according to any one of claims 86-102, wherein the extrusion process has a residence time of less than 5 minutes.

104. The method of claim 103, wherein the residence time is less than 2 minutes.

105. The method of claim 104, wherein the residence time is less than 1 minute.

106. The method according to any one of claims 86-105, wherein the extrusion process comprises an observed torque from about 20 Gm to about 200 Gm.

107. The method of claim 106, wherein the observed torque is from about 50 Gm to about 150 Gm.

108. The method of claim 107, wherein the observed torque is from about 60 Gm to about 100 Gm.

109. The method according to any one of claims 86-108, wherein the pharmaceutical composition comprises an amorphous active pharmaceutical ingredient.

110. The method according to any one of claims 86-108, wherein the pharmaceutical composition comprises a semi-crystalline active pharmaceutical ingredient.

111. The method according to any one of claims 86-108, wherein the pharmaceutical composition comprises a crystalline active pharmaceutical ingredient.

112. The method according to any one of claims 86-111, wherein the active pharmaceutical ingredient is absorbed on the first absorbent or the second absorbent.

113. The method of claim 112, wherein the active pharmaceutical ingredient is absorbed on the first absorbent.

114. The method of claim 112, wherein the active pharmaceutical ingredient is absorbed on the second absorbent.

115. The method according to any one of claims 112-114, wherein the absorbed active pharmaceutical ingredient causes the first absorbent or the second absorbent to form an agglomeration.

116. The method according to any one of claims 86-115, wherein the active pharmaceutical ingredient and the first absorbent are homogenously mixed.

117. The method according to any one of claims 86-115, wherein the active pharmaceutical ingredient and the second absorbent are homogenously mixed.

118. The method according to any one of claims 86-117, wherein the first absorbent and the second absorbent are homogenously mixed.

119. The method according to any one of claims 86-118, wherein the active pharmaceutical ingredient, the first absorbent, and the second absorbent are homogenously mixed.

120. The method according to any one of claims 86-119, wherein the active pharmaceutical ingredient is a poorly soluble drug.

121. The method according to any one of claims 86-120, wherein the active pharmaceutical ingredient is a BCS class 1 drug.

122. The method according to any one of claims 86-120, wherein the active pharmaceutical ingredient is a BCS class 2 drug.

123. The method according to any one of claims 86-120, wherein the active pharmaceutical ingredient is a BCS class 3 drug.

124. The method according to any one of claims 86-120, wherein the active pharmaceutical ingredient is a BCS class 4 drug.

125. The method according to any one of claims 86-124, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintic, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytic, lipid regulating agents, leukotriene inhibitors, macrolides, muscle relaxants, neuroleptics, nutritional agents, opioid analgesics, protease inhibitors, or sedatives.

126. The method according to any one of claims 86-125, wherein the pharmaceutical composition comprises from about 5% w/w to about 90% w/w of the active pharmaceutical ingredient.

127. The method according to any one of claims 86-126, wherein the pharmaceutical composition comprises from about 10% w/w to about 80% w/w of the active pharmaceutical ingredient.

128. The method according to any one of claims 86-127, wherein the pharmaceutical composition comprises from about 20% w/w to about 60% w/w of the active pharmaceutical ingredient.

129. The method according to any one of claims 86-127, wherein the pharmaceutical composition comprises from about 10% w/w to about 40% w/w of the active pharmaceutical ingredient.

130. The method according to any one of claims 86-127, wherein the pharmaceutical composition comprises from about 40% w/w to about 80% w/w of the active pharmaceutical ingredient.

131. The method according to any one of claims 86-130, wherein the first absorbent is a silicate.

132. The method of claim 131, wherein the silicate is a silicate salt.

133. The method of claim 132, wherein the silicate is an aluminum silicate.

134. The method according to any one of claims 131-133, wherein the silicate is magnesium aluminum silicate.

135. The method according to any one of claims 86-134, wherein the pharmaceutical composition comprises from about 2.5% w/w to about 45% w/w of the first absorbent.

136. The method according to any one of claims 86-135, wherein the pharmaceutical composition comprises from about 5% w/w to about 40% w/w of the first absorbent.

137. The method according to any one of claims 86-136, wherein the pharmaceutical composition comprises from about 10% w/w to about 25% w/w of the first absorbent.

138. The method according to any one of claims 86-136, wherein the pharmaceutical composition comprises from about 30% w/w to about 40% w/w of the first absorbent.

139. The method according to any one of claims 86-138, wherein the second absorbent is silica or aluminum comprising a plurality of pores.

140. The method according to any one of claims 86-139, wherein the second absorbent is silica.

141. The method according to any one of claims 86-140, wherein the second absorbent is silica comprising a plurality of pores, wherein the pores comprise a diameter between about 0.1 nm and about 50 nm.

142. The method of claim 141, wherein the pores have a diameter between 2 nm and about 50 nm.

143. The method according to any one of claims 86-142, wherein the pharmaceutical composition comprises from about 2.5% w/w to about 45% w/w of the second absorbent.

144. The method according to any one of claims 86-143, wherein the pharmaceutical composition comprises from about 5% w/w to about 40% w/w of the second absorbent.

145. The method according to any one of claims 86-144, wherein the pharmaceutical composition comprises from about 10% w/w to about 25% w/w of the second absorbent.

146. The method according to any one of claims 86-144, wherein the pharmaceutical composition comprises from about 30% w/w to about 40% w/w of the second absorbent.

147. The method according to any one of claims 86-146, wherein the pharmaceutical composition comprises the same amount of the first absorbent and the second absorbent.

148. The method according to any one of claims 86-147, wherein the surfactant is a polysorbate derivative.

149. The method of claim 148, wherein the surfactant is poly(ethylene glycol) derivatized polysorbate.

150. The method of claim 149, wherein the surfactant comprises from about 10 to about 30 poly(ethylene glycol) repeating units.

151. The method of claim 150, wherein the surfactant comprises 20 poly(ethylene glycol) repeating unit.

152. The method according to any one of claims 148-151, wherein the surfactant comprises a fatty acid.

153. The method of claim 152, wherein the fatty acid is oleic acid.

154. The method according to any one of claims 86-153, wherein the pharmaceutical composition comprises from about 0.5% w/w to about 20% w/w of the surfactant.

155. The method according to any one of claims 86-154, wherein the pharmaceutical composition comprises from about 1% w/w to about 10% w/w of the surfactant.

156. The method according to any one of claims 86-155, wherein the pharmaceutical composition comprises from about 2.5% w/w to about 7.5% w/w of the surfactant.

157. The method according to any one of claims 86-156, wherein the pharmaceutical composition comprises an excipient.

158. The method of claim 157, wherein the excipient is a laser absorbing species.

159. The method according to any one of claims 86-158, wherein the pharmaceutical composition comprises a second active pharmaceutical ingredient.

160. The method according to any one of claims 86-159, wherein the pharmaceutical composition comprises a pharmaceutically acceptable polymer.

161. The method according to any one of claims 86-160, wherein the pharmaceutical composition is substantially free of any other compound.

162. The method according to any one of claims 86-161, wherein the pharmaceutical composition is essentially free of any other compound.

163. The method according to any one of claims 86-162, wherein the pharmaceutical composition is entirely free of any other compound.

164. The method according to any one of claims 86-163, wherein the pharmaceutical composition is substantially free of any other compound other than the active pharmaceutical ingredient, the first absorbent, the second absorbent, an excipient, a second active pharmaceutical ingredient, or a pharmaceutically acceptable polymer.

165. The method according to any one of claims 86-164 further comprising subjecting the pharmaceutical composition to milling.

166. The method according to any one of claims 86-165 further comprising formulating the pharmaceutical composition into a unit dose.

167. The method of claim 166, wherein the unit dose is formulated for oral delivery.

168. The method of claim 167, wherein the oral delivery is formulated as a tablet, capsule, or suspension.

169. The method according to any one of claims 86-168, wherein the pharmaceutical composition comprises a Carr's Index from about 5 to about 25.

170. The method according to any one of claims 86-169, wherein the Carr's Index is from about 5 to about 15.

171. The method according to any one of claims 86-170, wherein the pharmaceutical composition comprises a surface area of greater than 100 m²/g.

172. The method of claim 171, wherein the surface area is greater than 200 m²/g.

173. The method of either claim 171 or claim 172, wherein the surface area is from about 100 m²/g to about 500 m²/g.

174. The method according to any one of claims 171-173, wherein the surface area is 150 m²/g to about 400 m²/g.

175. The method according to any one of claims 86-174, wherein the pharmaceutical composition comprises a mean or average particle size distribution of greater than about 25 μm.

176. The method of claim 175, wherein the mean or average particle size distribution is greater than about 50 μm.

177. The method of claim 175, wherein the mean or average particle size distribution is from about 25 μm to about 500 μm.

178. The method according to any one of claims 175-177, wherein the mean or average particle size distribution is from about 50 μm to about 250 μm.

179. The method according to any one of claims 175-178, wherein the mean or average particle size distribution is from about 60 μm to about 100 μm.

180. The method according to any one of claims 86-179, wherein the pharmaceutical composition has a flowability as a function of angle of repose of less than about 40.

181. The method of claim 180, wherein the flowability is from about 5 to about 40.

182. The method of either claim 180 or claim 181, wherein the flowability is from about 15 to about 35.

183. The method according to any one of claims 180-182, wherein the flowability is from about 20 to about 30.

184. The method according to any one of claims 86-183, wherein the pharmaceutical composition comprises a drug content uniformity of greater than about 75%.

185. The method of claim 184, wherein the drug content uniformity is greater than 80%.

186. The method of either claim 184 or claim 185, wherein the drug content uniformity is from about 90% to about 110%.

187. The method according to any one of claims 184-186, wherein the drug content uniformity is from about 95% to about 105%.

188. The method according to any one of claims 86-187, wherein the pharmaceutical composition is formulated as granules.

189. The method according to any one of claims 86-188, wherein the pharmaceutical composition comprises:
(A) about 20% w/w to about 60% w/w indomethacin;
(B) about 17.5% w/w to about 37.5% w/w magnesium aluminum silicate;
(C) about 17.5% w/w to about 37.5% w/w porous silica; and
(D) about 5% w/w of Tween® 80.

190. The method according to any one of claims 86-188, wherein the pharmaceutical composition comprises:
(A) about 20% w/w to about 80% w/w mefenamic acid;
(B) about 7.5% w/w to about 37.5% w/w magnesium aluminum silicate;
(C) about 7.5% w/w to about 37.5% w/w porous silica; and
(D) about 5% w/w of Tween® 80.

191. A method of preparing a unit dose comprising:
(A) obtaining a pharmaceutical composition according to any one of claims 1-84; and
(B) subjecting the pharmaceutical composition to an additive manufacturing process to obtain a unit dose.

192. The method of claim 191, wherein the additive manufacturing process is a 3D printing process.

193. The method of either claim 191 or 192, wherein the additive manufacturing process is an additive manufacturing layer process.

194. The method according to any one of claims 191-193, wherein the additive manufacturing process is selective layer sintering.

195. The method according to any one of claims 191-194, wherein the unit dose is formulated in a manner to be directly administered to a patient without further processing.

196. A pharmaceutical composition prepared for the methods described in any one of claims 69-195.

197. A method of treating a disease or disorder in a patient in need thereof comprising administering to the patient a therapeutically effective amount of a pharmaceutical composition according to any one of claims 1-85 and 196, wherein the active pharmaceutical ingredient is effective to treat the disease or disorder.
